(19) United States
(12) Patent Application Publication
(10) Pub. No.: US 2026/0137510 A1
(43) Pub. Date: May 21, 2026

(54) PROSTHETIC VALVES AND DELIVERY ASSEMBLIES WITH POSITIONING ARMS

(71) Applicant: Edwards Lifesciences Corporation, Irvine, CA (US)

(72) Inventors: Tal Reich, Moledet (IL); Tamir S. Levi, Zikhron Yaakov (IL)

(73) Assignee: Edwards Lifesciences Corporation, Irvine, CA (US)

(21) Appl. No.: 19/444,034

(22) Filed: Jan. 8, 2026

Related U.S. Application Data
(63) Continuation of application No. PCT/US2024/038513, filed on Jul. 18, 2024.
(60) Provisional application No. 63/528,036, filed on Jul. 20, 2023.

(51) Int. Cl. A61F 2/24

(52) U.S. Cl. CPC A61F 2/2418; A61F 2/2436

(57) ABSTRACT

The present disclosure relates to positioning arms of a prosthetic valve or a delivery apparatus thereof, utilized to assist in proper axial positioning of the prosthetic valve relative to coronary ostia. In an example, a prosthetic valve comprises a frame, a plurality of leaflets mounted inside the frame, and one or more positioning arms affixed at bases thereof to the frame and extending proximally therefrom to terminate at free ends which are biased, in a free state of the positioning arms, away from the frame, to a position that can be proximal to the leaflets, configured to engage with ceilings of the Valsalva sinuses during an implantation procedure of the prosthetic valve.

### CROSS-REFERENCE TO RELATED APPLICATIONS

This application is a continuation of International Application No. PCT/US2024/038513, filed July 18, 2024, which claims the benefit of U.S. Provisional Application No. 63/528,036, filed July 20, 2023, which is incorporated by reference herein.

### FIELD

The present disclosure relates to prosthetic valves and delivery assemblies equipped with positioning arms, and to methods of utilization thereof.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient’s vasculature (for example, through a femoral artery and the aorta) to the heart. The prosthetic heart valve is positioned within the native valve and expanded to its functional size.

### SUMMARY

Certain types of prosthetic valves may pose a risk of obstructing blood flow to branched arteries at the region of implantation. Aortic valvular sinuses of a native aortic valve have two coronary ostia. A prosthetic valve implanted inside a native aortic valve can block access to at least one of the coronary arteries, for example if the valve includes components that may prevent blood flow across the frame, along regions that may be aligned with, or extend past, the coronary ostia. Thus, it is desirable to provide devices and methods by which a prosthetic valve can be positioned relative to the native annulus in a manner that prevents coronary artery obstruction.

According to some aspects of the disclosure, there is provided a prosthetic valve comprising a frame, a valvular structure mounted within the frame, and at least one positioning arm. The frame is movable between a radially compressed state and a radially expanded state. The valvular structure comprises a plurality of leaflets configured to regulate flow through the prosthetic valve. The at least one positioning arm extends proximally from a base affixed to the frame, to a free end. The at least one positioning arm is configured to transition between a compacted state and a free state and is biased to the free state in which the free end is radially spaced from the frame and is distal to a proximal end of the frame.

In some examples, the base can be proximal to a distal end of the frame.

In some examples, the free end can be proximal to the valvular structure in the free state.

In some examples, each leaflet can comprise a free edge, wherein the free end is proximal to free edges of the leaflets.

In some examples, the valvular structure can comprise commissures coupled to commissure attachment features of the frame, wherein the free end is proximal to commissures in the free state.

In some examples, the axial distance between the free end of the at least one positioning member and the commissures can be greater than 2 mm.

In some examples, the free end can be configured to engage with a sinus ceiling of a Valsalva sinus.

In some examples, when the free end is engaged with the sinus ceiling, an axial gap can be defined between an STJ level and the valvular structure.

According to some aspects of the disclosure, there is provided a method of implanting a prosthetic valve, the method comprising advancing a delivery assembly that comprises a delivery apparatus carrying a prosthetic valve, retained in a radially compressed state inside a capsule of the delivery apparatus, to a native heart valve. The method further comprises exposing at least a portion of an inflow section of the prosthetic valve and one or more positioning arms affixed to a frame of the prosthetic valve, such that free ends of the positioning arms spring outwardly towards Valsalva sinuses of the native heart valve, while at least a portion of an outflow section of the prosthetic valve remains retained inside the capsule. The method further comprises adjusting the axial position of the prosthetic valve such that the free ends of the positioning arms engage with sinus ceilings of the Valsalva sinuses. The method further comprises exposing the remainder of the prosthetic valve from the capsule.

In some examples, each of the one or more positioning arms can extend proximally from a base at which it is affixed to the frame, to the free end.

In some examples, the base is proximal to a distal end of the frame.

In some examples, the adjusting the axial position can comprise pressing the free ends against the sinus ceilings.

In some examples, the exposing the remainder of the prosthetic valve can comprise anchoring the inflow section against a native annulus of the native heart valve.

In some examples, the exposing the remainder of the prosthetic valve can comprise anchoring the outflow section against an ascending aorta proximal to the Valsalva sinuses.

According to some aspects of the disclosure, there is provided a delivery assembly comprising a prosthetic valve and a delivery apparatus. The prosthetic valve comprises a frame which is movable between a radially compressed state and a radially expanded state, and a valvular structure mounted within the frame and comprising a plurality of leaflets configured to regulate flow through the prosthetic valve. The delivery apparatus comprises a delivery shaft, a first capsule, a second capsule, and at least one positioning arm. The first capsule is at a distal end of the delivery shaft. The second capsule is axially movable relative to the first capsule. The at least one positioning arm extends proximally from a base affixed to the first capsule, to a free end. The at least one positioning arm is configured to transition between a compacted state and a free state and is biased to the free state in which the free end is radially spaced from the first capsule.

In some examples, the frame can comprises an inflow section and an outflow section having a diameter greater than that of the inflow section in the expanded state.

In some examples, wherein the first capsule can be configured to retain therein at least a portion of the outflow section in the compressed state.

In some examples, the free end can be proximal to a proximal end of the frame, in the free state of the positioning arm, when the outflow portion is retained inside the first capsule.

In some examples, the free end can be distal to free edges of the leaflets, in the free state of the positioning arm, when the outflow portion is retained inside the first capsule.

In some examples, the free end can be distal to commissures of the valvular structure, in the free state of the positioning arm, when the outflow portion is retained inside the first capsule.

In some examples, the base can be affixed to a distal end portion of the first capsule.

In some examples, the second capsule can comprise a second capsule proximal portion and a second capsule distal portion, wherein the second capsule proximal portion can be configured to cover at least part of the first capsule.

In some examples, the second capsule proximal portion can be configured to cover at least part of the at least one positioning arm.

In some examples, the second capsule proximal portion can be configured to maintain the at least one positioning arm in the compacted state when the second capsule proximal portion covers the positioning arm.

In some examples, the at least one positioning arm can be configured to spring outwards to the free state when uncovered from the second capsule.

In some examples, the delivery apparatus can further comprise at least one tensioning member attached to the free end of the at least one positioning arm, and extending proximally therefrom.

In some examples, the positioning arm can be configured to transition to the compacted state when the tensioning member attached thereto is tensioned.

In some examples, the tensioning member can extend from the free end of the positioning arm it is attached to, through a corresponding side opening formed at the first capsule, into the first capsule.

In some examples, the tensioning member can extend from the free end of the positioning arm it is attached to, through a corresponding side opening formed at the delivery shaft, into the delivery shaft.

According to some aspects of the disclosure, there is provided a method of implanting a prosthetic valve, the method comprising advancing a delivery assembly that comprises a delivery apparatus carrying a prosthetic valve having a frame retained in a radially compressed state inside a first capsule and a second capsule of the delivery apparatus, to a native heart valve, while one or more positioning arms attached at bases thereof to the first capsule are retained in a compacted state between the first capsule and a second capsule proximal portion disposed over the positioning arms. The method further comprises uncovering the positioning arms from the second capsule proximal portion, such that free ends of the positioning arms spring outwardly towards Valsalva sinuses of the native heart valve, while at least a portion of an outflow section of the prosthetic valve remains retained inside the first capsule. The method further comprises adjusting the axial position of the first capsule such that the free ends of the positioning arms engage with sinus ceilings of the Valsalva sinuses. The method further comprises exposing the prosthetic valve such that it is allowed to expand against the native aortic valve.

In some examples, the positioning arms extend proximally from the bases to the free ends.

In some examples, the uncovering the positioning arms comprises axially moving the second capsule and the first capsule away from each other.

In some examples, the adjusting the axial position comprises pressing the free ends against the sinus ceilings.

In some examples, the method can further comprise, after the adjusting the axial position of the second capsule and prior to the exposing the prosthetic valve, transitioning the positioning arms to the compacted state.

In some examples, the transitioning the positioning arms to the compacted state can comprise applying tension to tensioning members coupled to the free ends of the positioning arms and extending proximally therefrom.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### In the Figures:

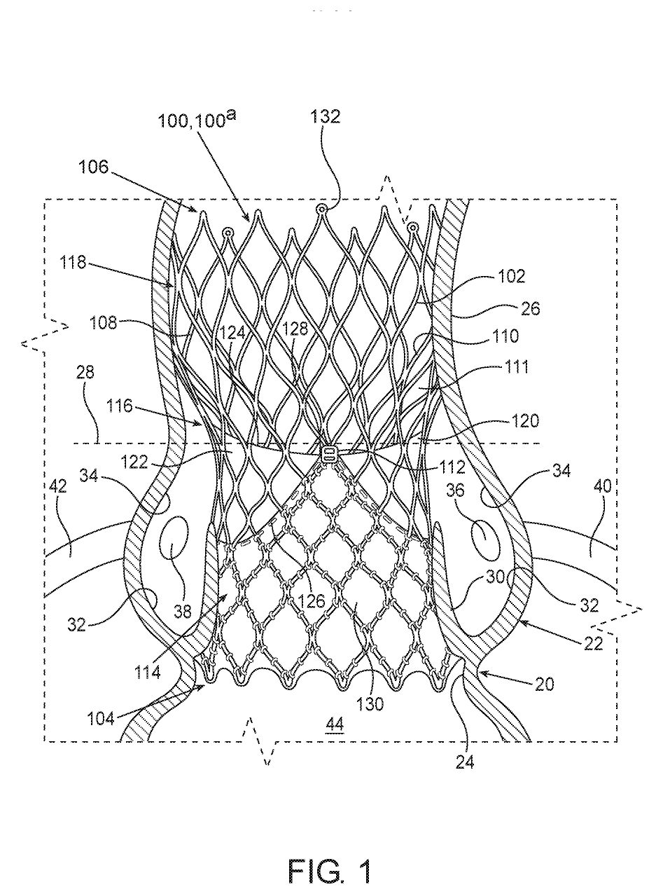

FIG. 1 shows an exemplary prosthetic valve implanted inside a native aortic valve.

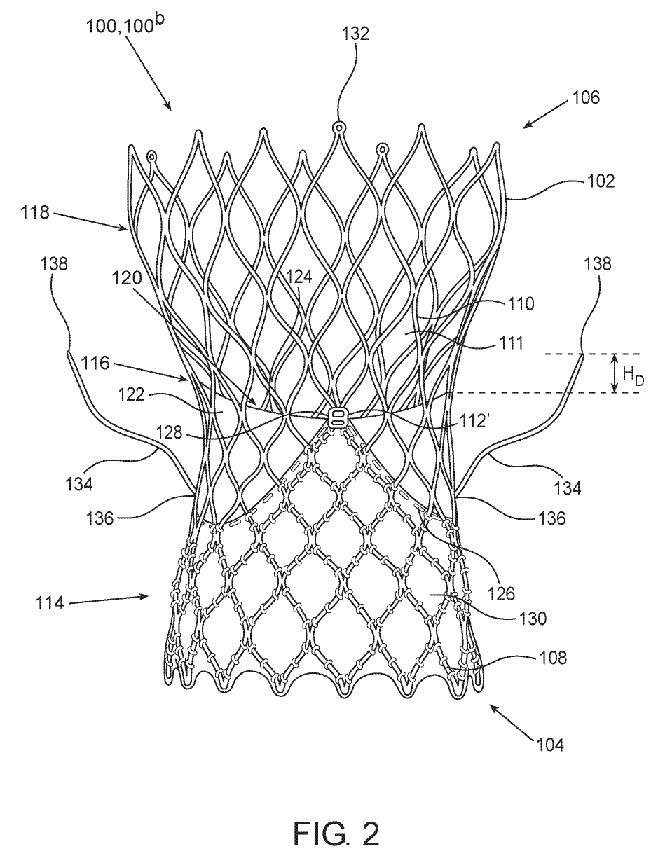

FIG. 2 shows an exemplary prosthetic valve equipped with positioning arms.

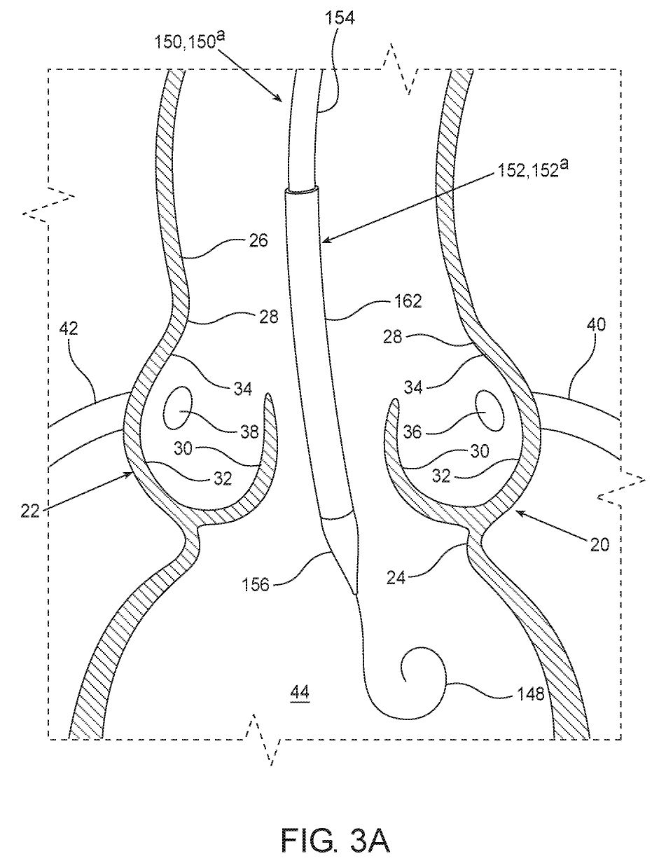

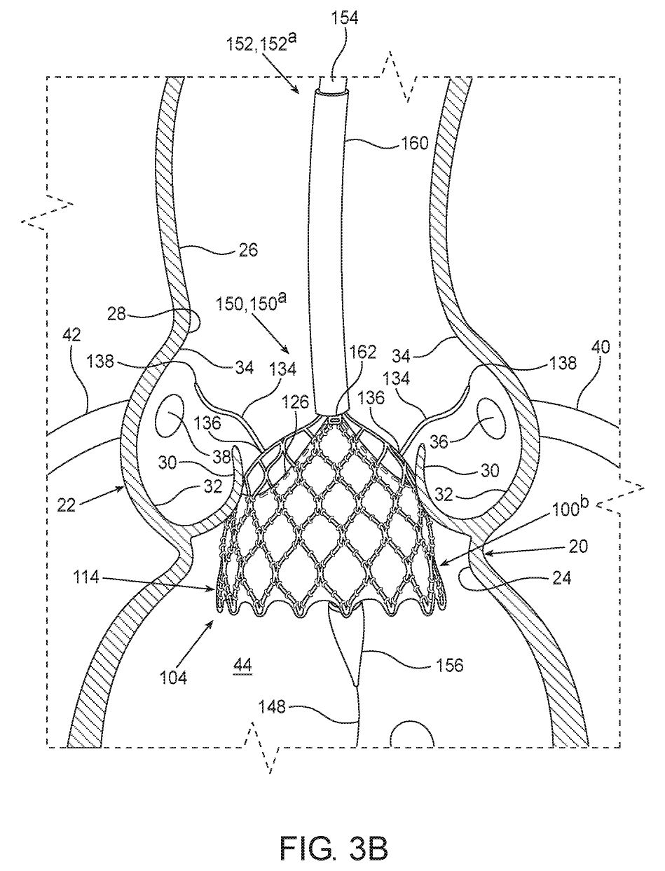

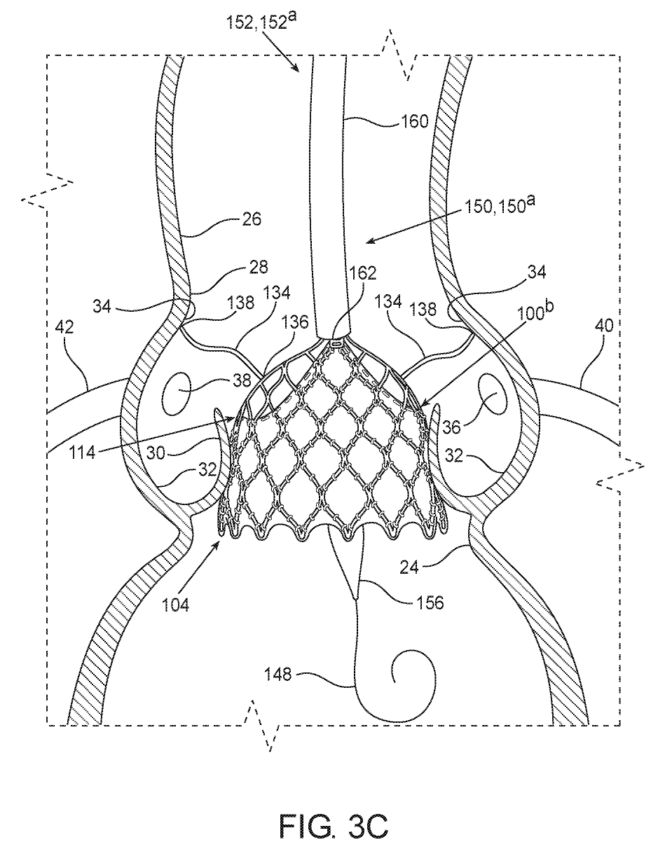

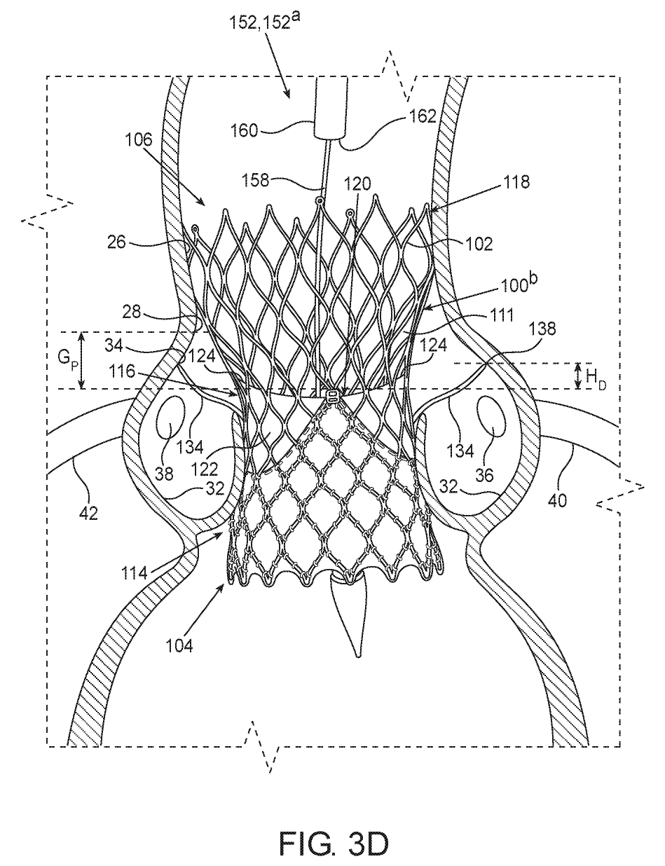

FIG. 3A shows a distal portion of an exemplary delivery assembly advanced towards a native aortic valve.

FIG. 3B shows an inflow section and positioning arms of the prosthetic valve of FIG. 2 exposed out of a capsule of the delivery assembly of FIG. 3A.

FIG. 3C shows the delivery assembly of FIG. 3B positioned such that the positioning arms engage with sinus ceilings of the native aortic valve.

FIG. 3D shows the prosthetic valve fully deployed out of the capsule of the delivery assembly of FIG. 3C.

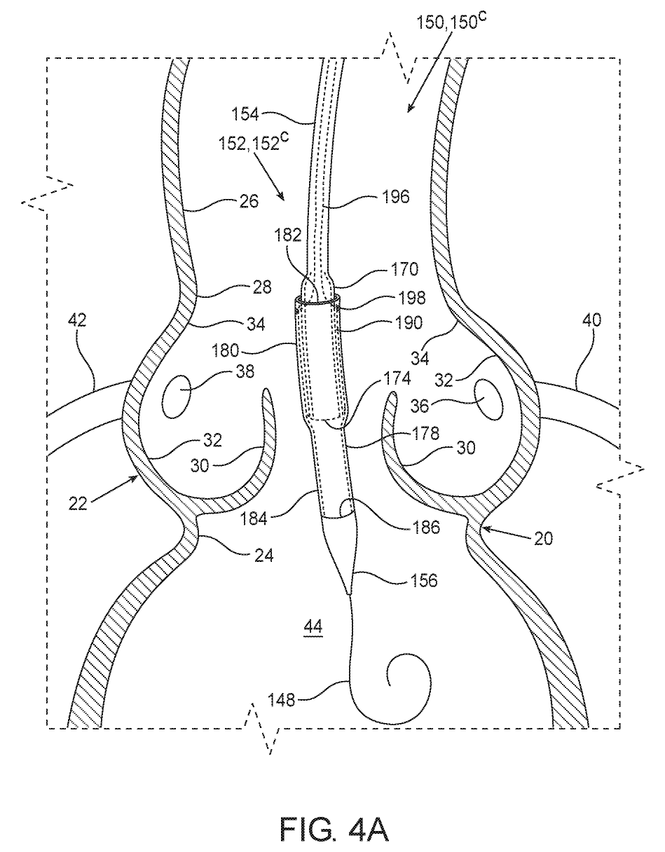

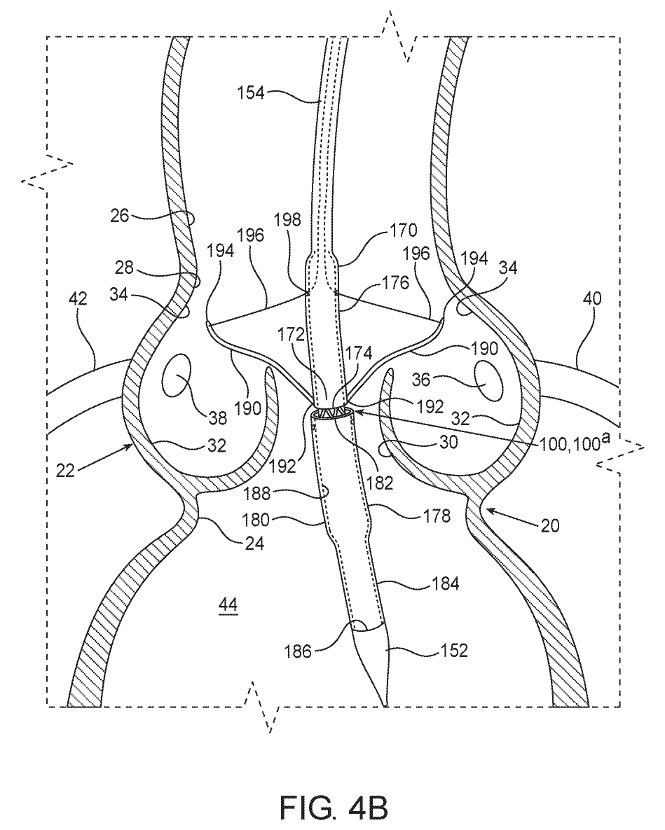

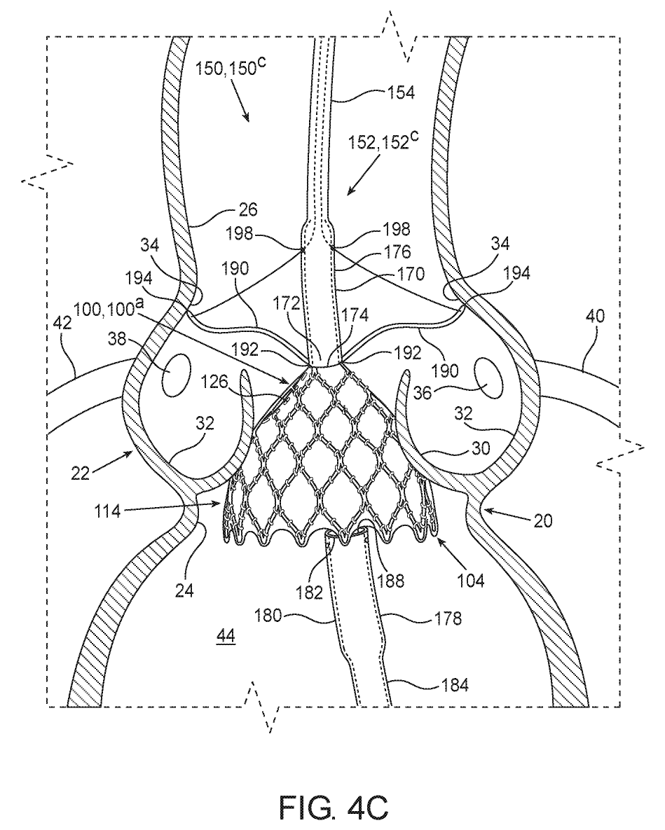

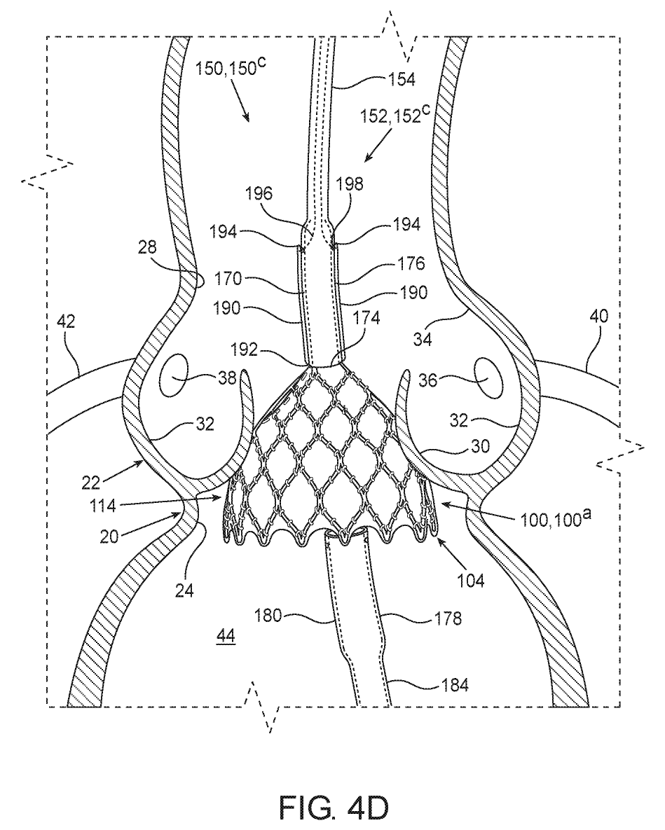

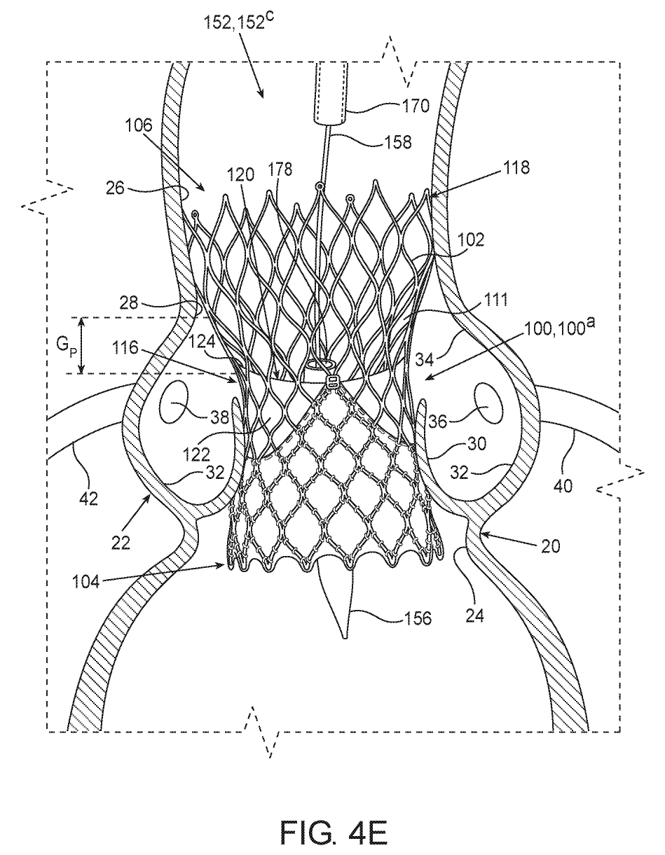

FIG. 4A shows a distal portion of an exemplary delivery assembly that includes a first capsule and a second capsule partially covering a portion of the first capsule, advanced towards a native aortic valve.

FIG. 4B shows the first capsule uncovered from the second capsule, exposing positioning arms attached to the first capsule.

FIG. 4C shows the delivery assembly of FIG. 4B positioned such that the positioning arms engage with sinus ceilings of the native aortic valve, and an inflow section of a prosthetic valve exposed out of the second capsule.

FIG. 4D shows the positioning arms of the delivery assembly of FIG. 4C transitioned to a compacted state.

FIG. 4E shows the prosthetic valve fully deployed out of the capsules of the delivery assembly of FIG. 4D.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like "provide" or "achieve" to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms "a", "an", and "the" include the plural forms unless the context clearly dictates otherwise. Additionally, the terms "have" or "includes" means "comprises". Further, the terms "coupled", "connected", and "attached", as used herein, are interchangeable and generally mean physically, mechanically, chemically, magnetically, and/or electrically coupled or linked and does not exclude the presence of intermediate elements between the coupled or associated items absent specific contrary language. As used herein, "and/or" means "and" or "or", as well as "and" and "or".

Directions and other relative references may be used to facilitate discussion of the drawings and principles herein, but are not intended to be limiting. For example, certain terms may be used such as "inner", "outer", "upper", "lower", "inside", "outside", "top", "bottom", "interior", "exterior", "left", right", and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an "upper" part can become a "lower" part simply by turning the object over. Nevertheless, it is still the same part and the object remains the same.

The term "plurality" or "plural" when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms "proximal" and "distal" are defined relative to the use position of a delivery apparatus. In general, the end of the delivery apparatus closest to the user of the apparatus is the proximal end, and the end of the delivery apparatus farthest from the user (for example, the end that is inserted into a patient’s body) is the distal end. The term "proximal" when used with two spatially separated positions or parts of an object can be understood to mean closer to or oriented towards the proximal end of the delivery apparatus. The term "distal" when used with two spatially separated positions or parts of an object can be understood to mean closer to or oriented towards the distal end of the delivery apparatus. The terms "longitudinal" and "axial" are interchangeable, and refer to an axis extending in the proximal and distal directions, unless otherwise expressly defined.

The terms "axial direction", "radial direction", and "circumferential direction" have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms "integrally formed" and "unitary" refer to a construction that does not include any welds, fasteners, or other means for securing separately formed pieces of material to each other.

As used herein, operations that occur "simultaneously" or "concurrently" occur generally at the same time as one another, although delays in the occurrence of operation relative to the other due to, for example, spacing between components, are expressly within the scope of the above terms, absent specific contrary language.

As used herein, terms such as "first", "second", and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term "substantially" means the listed value and/or property and any value and/or property that is at least 75% of the listed value and/or property. Equivalently, the term "substantially" means the listed value and/or property and any value and/or property that differs from the listed value and/or property by at most 25%. For example, "at least substantially parallel" refers to directions that are fully parallel, and to directions that diverge by up to 22.5 degrees.

In the present disclosure, a reference numeral that includes an alphabetic label (for example, "a", "b", "c", etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

FIG. 1 shows an exemplary prosthetic valve 100 that can be implanted in a native heart valve, such as the native aortic valve 20. The aortic root 22 shown in FIG. 1 to extend between the left ventricle 44 and the ascending aorta 26, includes the native aortic valve 20 and a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported by the aortic annulus 24, which is a ring of fibrous tissue at the transition point between the left ventricle 44 and the aortic root 22. In a normally functioning native valve 20, the leaflets 30 can cycle between open and closed positions to regulate flow of blood from the left ventricle 44 to the ascending aorta 26. The aortic root 22 further comprises the sinuses of Valsalva 32, which are three bulges located between the leaflets 30 and the of the aortic wall, with two of the Valsalva sinuses 32 communicating the coronary arteries 40, 42. The coronary artery ostia 36, 38 are the openings that connect the aortic root 22 to the coronary arteries 40, 42. The Valsalva sinuses 32 are limited proximally by the attachments of the leaflets 30 and distally by sinus ceilings 34 terminating at the level of the sinotubular junction (STJ) 28.

The term "prosthetic valve", as used herein, refers to any type of a prosthetic valve deliverable to a patient's target site over a catheter, which is radially expandable and compressible between a radially compressed, or crimped, state, and a radially expanded state. Thus, the prosthetic valve can retained by a delivery apparatus (such as delivery apparatus 152 shown in FIGS. 3A-3D, for example) in the radially compressed state during delivery, and then expanded (or allowed to passively expand) to the radially expanded state once the prosthetic valve reaches the implantation site. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule is withdrawn proximally relative to the prosthetic valve. Mechanically expandable valves are a category of prosthetic valves that rely on a mechanical actuation mechanism for expansion.

FIG. 1 shows an example of a prosthetic valve 100, which can be a self-expandable valve or any other type of valve, illustrated in an expanded state. The prosthetic valve 100 comprises an annular frame 102 movable between a radially compressed configuration and a radially expanded configuration, and a valvular structure 120 that comprises prosthetic valve leaflets 122 mounted within the frame 102. In the case of a self-expandable valve 100, the frame 102, which can also be referred to as "a stent" or "a support structure", may be self-expanding, configured to expand from a compressed state to an expanded state under the inherent resiliency or elastic force of the frame.

The frame 102 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term "outflow", as used herein, refers to a region of the prosthetic valve through which the blood flows through and out of the prosthetic valve 100.

The term "inflow", as used herein, refers to a region of the prosthetic valve through which the blood flows into the prosthetic valve 100.

In the context of the present application, the terms "lower" and "upper" are used interchangeably with the terms "inflow" and "outflow", respectively. Thus, for example, the lower end of the prosthetic valve is its inflow end and the upper end of the prosthetic valve is its outflow end.

In the context of the present application, the terms "lower" and "upper" are used interchangeably with the terms "distal to" and "proximal to", respectively. Thus, for example, a lowermost component can refer to a distal-most component, and an uppermost component can similarly refer to a proximal-most component.

The terms "longitudinal" and "axial", as used herein, refer to an axis extending in the proximal and distal directions, unless otherwise expressly defined.

In some examples, the frame can be formed of a shape memory material (e.g., a nickel titanium alloy, such as Nitinol) such that the frame can be shape-set to a particular configuration and then elastically deformed to one or more other configurations. As one example, the frame 102 can be formed of Nitinol and shape-set in the radially-expanded state. The frame 102 can be elastically deformed to the radially-compressed state prior to delivery into the patient's body, and allowed to self-expand when an enclosure in which the valve is retained during delivery, such as a capsule, is removed from the frame upon reaching the site of implantation.

In some examples, the frame can be formed of a plastically-deformable material (e.g., stainless steel or cobalt chromium alloy) such that the frame can be formed in a particular configuration and then plastically deformed to one or more configurations which are radially smaller or larger than the configuration in which the frame is formed.

In some examples, the frame 102 can be formed from a single piece of material (e.g., a metal tube). This can be accomplished, for example, via laser cutting, electroforming, and/or physical vapor deposition. Nevertheless, in some examples, the frame can be constructed by forming individual components and coupling the individual components together (e.g., via welding, brazing, and/or other means for bonding).

The frame 102 includes a plurality of intersecting struts 108. A strut 108 may be any elongated member or portion of the frame 102. The frame 102 can include a plurality of strut rungs that can collectively define one or more rows of cells 110. At least some of the struts 108 can be pivotable or bendable relative to each other, so as to permit frame expansion or compression

In the example illustrated in FIG. 1, the frame 102 is shown to include multiple levels, including an inflow section 114 extending proximally from the inflow end 104, an enlarged or flared outflow section 118 extending distally from the outflow end 106, and a transition section 116 between the inflow section 114 and the outflow sections 118. As shown, the outflow section 118 can have a larger cross-section or diameter than that of the inflow section 114 in the expanded state of the prosthetic valve 100, while the transition section 116 may taper outwardly from the inflow section 114 to the outflow section 118. When implanted inside a native aortic valve 20, the inflow section 114 can extend into and anchor against the aortic annulus 24, and the outflow section 118, which can be also referred to as the aortic section, can be positioned in the ascending aorta 26. In some examples, implantation of the prosthetic valve 100 can be performed such that a portion of the inflow section 114 extends into the left ventricle 44, while the outflow section 118 is anchored against the ascending aorta 26 above the STJ level 28.

A valvular structure 120 of the prosthetic valve 100 can include a plurality of prosthetic valve leaflets 122 (for example, three leaflets), positioned at least partially within the frame 102, and configured to regulate flow of blood through the prosthetic valve 100 from the inflow end 104 to the outflow end 106. While three leaflets 122 arranged to collapse in a tricuspid arrangement, are shown in the illustrated example, it will be clear that a prosthetic valve 100 can include any other number of leaflets 122. Adjacent leaflets 122 can be arranged together to form commissures 128 that are coupled (directly or indirectly) to respective portions of the frame 102, thereby securing at least a portion of the valvular structure 120 to the frame 102. The prosthetic valve leaflets 122 can be made from, in whole or part, biological material (for example, pericardium), bio-compatible synthetic materials, or other such materials.

In some examples, the frame 102 can include commissure attachment features 112 for mounting the commissures 128 of the valvular structure 120 to the frame 102. In some examples, each commissure attachment feature 112 and can include one or more eyelets, configured to facilitate suturing of the commissures 128 to the frame 102. As illustrated in FIG. 1, each commissure attachment feature 112 can lay at the intersection of four cells 110, two of the cells 110 being adjacent one another in the same annular row of cells, and the other two cells 110 being in different annular rows and lying in end to end relationship. The commissure attachment features 112 may be positioned entirely within the inflow section 114 or at the juncture of inflow section 114 and transition section 116.

In some examples, the commissure attachment features 112 are defined as selected cells 110 of the frame 102, such that each commissure 128 spans the corresponding cell opening 111 and is attached, such as by sutures, to the struts 108 defining the corresponding cell 110 (examples not shown). In some examples, the commissure attachment features 112 can be defined along vertical struts of the frame to which the commissures 128 are attached (examples not shown). In some examples, selected vertical struts of the frame can include commissure windows (not shown) through which tabs or flaps of the leaflets can extend.

In some examples, the prosthetic valve 100 can comprise at least one skirt or sealing member. For example, the prosthetic valve 100 can include an inner skirt 130 which can be secured to the inner surface of the frame 102. Such an inner skirt can be configured to function, for example, as a sealing member to prevent or decrease perivalvular leakage. An inner skirt 130 can further function as an anchoring region for leaflets 122 to the frame 102, and/or function to protect the leaflets 122 against damage which may be caused by contact with the frame 102, for example during valve crimping or during working cycles of the prosthetic valve 100. An inner skirt 130 can be disposed around and attached to the inner surface of frame 102, while the leaflets 122 can be sutured or otherwise coupled to the inner skirt 130 along a scalloped line 126. Each leaflet can extend from a cusp edge (not indicated separately) along which the leaflet 122 can be attached to the skirt 130, and an opposite free edge 124 at an outflow end of the leaflet 122, wherein the free edges 124 can move towards each other to coapt during diastolic phases of the cardiac cycles, and away from each other, towards the frame 102, during the systolic phases. An inner skirt 130 can be coupled to the frame 102 via sutures or another form of coupler.

The prosthetic valve 100 can comprise, in some examples, an outer skirt (not shown) mounted on the outer surface of frame 102, configured to function, for example, as a sealing member retained between the frame 102 and the surrounding tissue of the native annulus against which the prosthetic valve is mounted, thereby reducing risk of paravalvular leakage (PVL) past the prosthetic valve 100. The outer skirt can be coupled to the frame 102 via sutures or another form of coupler.

Any of the inner skirt 130 and/or outer skirt can be made of various suitable biocompatible materials, such as, but not limited to, various synthetic materials (for example, PET) or natural tissue (for example pericardial tissue). In some cases, the inner skirt 130 can be formed of a single sheet of material that extends continuously around the inner surface of frame 102. In some cases, the outer skirt can be formed of a single sheet of material that extends continuously around the outer surface of frame 102.

The cells 110, defined by interconnected struts 108, define cell openings 111. While some of the cell openings 111 can be covered by the inner skirt 130 and/or the outer skirt, at least a portion of the cell openings 111 can remain uncovered, such as cell openings 111 along the outflow section 118 of the valve 100. In some examples, the cells 110 of the outflow section 118 larger cells opening 111 than those defined by cells 110 of the inflow section 114. The larger cells 110 can be sized to allow blood to flow therethrough towards and into the coronary ostia 36, 38, and to optionally allow passage of a coronary catheter therethrough to preserve access to the coronary arteries 40, 42.

In some examples, the frame 102 can include one or more retaining elements 132 at the outflow end 106. The retaining elements 132 can be sized and shaped to cooperate with retaining structures (not shown) of a delivery apparatus. The engagement of the retaining elements 132 with the retaining structures of the delivery apparatus may help maintain the prosthetic valve 100 in assembled relationship with the delivery apparatus, minimize longitudinal movement of the prosthetic valve relative to the delivery apparatus during unsheathing or re-sheathing procedures, and help prevent rotation of the prosthetic valve relative to the delivery apparatus as the delivery apparatus is advanced to the target location and during deployment. The retaining elements 132 can include eyelets, or can be otherwise formed such as by including proximal enlarged or T-shaped structures.

A key factor in prosthetic valve deployment during implantation in a native aortic valve is properly positioning the prosthetic valve, e.g., accurately positioning the prosthetic valve relative to the coronary ostia. FIG. 1 shows a hypothetical coronary access obstruction that could occur in some cases from implantation of a prosthetic valve 100 within the native aortic valve 20 such that the valvular structure 120 spans a significant height of the Valsalva sinuses, with the outflow end of the valvular structure 120 positioned at, proximal to, or distal but in close proximity to, the STJ level 28. This in turn may compromise the ability for future access into the coronary arteries 40, 42 or perfusion through the frame 102 to the coronary arteries 40, 42 during the diastole phase of the cardiac cycle. The risk of compromising access or flow to the left coronary ostium 36 tends to be greater than obstructing the right coronary ostium 38 because the left coronary ostium 36 typically sits lower than the right coronary ostium 38.

FIG. 2 illustrates an exemplary prosthetic valve 100^{b}. Various examples for prosthetic valves 100, delivery assemblies 150 (shown, for example, in FIGS. 3A-3D), and/or components thereof, can be referred to, throughout the specification, with superscripts, for ease of explanation of features that refer to such exemplary examples. It is to be understood, however, that any reference to structural or functional features of any assembly, device or component, without a superscript, refers to these features being commonly shared by all specific exemplary examples that can be also indicated by superscripts. In contrast, features emphasized with respect to an exemplary implementation of any assembly, device or component, including prosthetic valve 100 and/or delivery assembly 150, referred to with a superscript, may be optionally shared by some but not necessarily all other exemplary examples. For example, both prosthetic valve 100^{a} shown in FIG. 1 and prosthetic valve 100^{b} shown in FIG. 2 are exemplary implementation of prosthetic valve 100, and thus can share features described for prosthetic valve 100 throughout the current disclosure, except that prosthetic valve 100^{b} further comprises one or more positioning arms 134, in contrast to prosthetic valve 100^{a} illustrated to exclude positioning arms 134 of the types described below.

As shown in FIG. 2, prosthetic valve 100 comprises one or more positioning arms 134 configured to assist in axial positioning during implantation inside the native aortic valve 20. While two positioning arms 134 are illustrated, it is to be understood that any other number is contemplated, including a single positioning arm, three positioning arms (for example, for placement in each of the three Valsalva sinuses 32), or more than three positioning arms. Any reference to a "positioning arm 134" in a singular form may refer to any plurality of positioning arms 134, and any reference to "positioning arms 134" in the plural form may similarly refer to a single positioning arm 134, unless stated otherwise.

The positioning arms 134 can be self-expandable arms that can be deployed into the sinuses 32. A positioning arm 134 extends from a base 136 at which it is attached to the frame 102, to a free end 138 which is biased radially away from the frame 102 to a position proximal to the base 136 and spaced away from the frame 102 in a free state of the positioning arm 134, referring to a state in which the positioning arm 134 is free to assume a pre-shaped configuration in the absence of external forces acting there-against.

In some examples, the positioning arms 134 are integrally formed with the frame 102. In some examples, the positioning arms 134 are formed as separate components affixed to the frame 102 at their bases 136, such as by welding, gluing, suturing, and the like. When a plurality of positioning arms 134 are provided, they can be equally or unequally spaced from each other around the circumference of the frame 102.

The positioning arms 134 can transition between a compacted state, in which the arms 134 can be pressed against the frame 102 (for example, in a compressed state of the prosthetic valve 100) and optionally assume a relatively elongated configuration that can be parallel to a longitudinal central axis of the frame 102, wherein the free ends 138 contact or are in close proximity to the frame 102, and a deployed or free state in which the arms 134 are allowed to extend away from the frame 102 such that the free ends 138 of the arms 134 are radially spaced from the frame 102. In some examples, the positioning arms 134 can be made of a shape memory material, such as a nickel-titanium alloy (for example, Nitinol), and are preset to a shape of the free state by heat treatment. The positioning arms 134 can be configured to assume a compacted state when constrained by a radial constrain of a delivery apparatus, and to be released to assume their free state after being released from the radial constrain, which can be performed prior to full expansion of the frame 102.

The positioning arms 134 proximally extend from the bases 136 towards the outflow end 106 such that the free ends 138 of the arms 134 are positioned proximal to the leaflets 122 of the valvular structure 120 in a free state of the arms 134. In some examples, the free ends 138 are proximal to the free edges 124 of the leaflets 122 in a free state of the arms 134. In some examples, the free ends 138 are proximal to the commissures 128. In some examples, the free ends 138 are proximal to the skirt 130 in a free state of the arms 134.

The positioning arms 134 are configured to engage with the sinuses 32 such that the free ends 138 can contact the sinus ceilings 34 when the arms 134 are deployed in the aortic valve 20. As mentioned above, a prosthetic valve 100 can include an outflow section 118 configured to extend into and engage with the ascending aorta 26, above the STJ level 28, while the free ends 138 are configured to contact the sinus ceilings 34 which are substantially at, or in close proximity to, the STJ level 28. Thus, in some examples, the free ends 138 of the arms 134 are proximal to the outflow end 106 of the frame 102 in a free state of the arms 134.

The positioning arms 134 of prosthetic valve 100^{b} are configured to assume their free or deployed state while the prosthetic valve 100^{b} is still at least partially compressed, in order to allow for adjustment of the axial position of the prosthetic valve 100^{b} and the arms 134 extending therefrom prior to full deployment of the valve 100^{b} inside the native aortic valve 20. Thus, it may be of advantage to position the arms 134 around the frame 102 in a position that will allow full deployment of the arms 134 while at least a portion of the prosthetic valve 100^{b} is retained in a compressed or partially compressed state.

FIGS. 3A-3D show a distal portion of an exemplary delivery assembly 150 used in a method for implantation of a prosthetic valve 100, such as prosthetic valve 100^{b}, within a native aortic valve 20. Delivery assembly 150 includes a delivery apparatus 152 and a prosthetic valve 100. The delivery apparatus 152 can include a handle (not shown) and an elongated delivery shaft 154 extending distally from the handle. The delivery apparatus 152 can also include an elongated nosecone shaft 158 extending distally from the handle through the delivery shaft 154. A nosecone 156 can be connected to the distal end of the nosecone shaft 158. The distal end portion of the delivery shaft 154 can be sized and shaped to house at least a portion of the prosthetic valve 100 in a radially compressed, delivery state during delivery of the prosthetic valve through, for example, the vasculature of a patient.

Although not illustrated, the handle of the delivery apparatus 152 can include one or more control mechanisms (such as knobs or other actuating mechanisms) for controlling different components of the delivery apparatus 152 in order to expand and/or deploy the prosthetic valve 100 and or components thereof, such as the positioning arms.

In some examples, a capsule can be attached to a distal end of the delivery shaft 154. Exemplary delivery apparatus 152^{a} of delivery assembly 150^{a} includes a capsule 160 terminating with a capsule distal end 162. Axial movement of the delivery shaft 154 in a distal direction relative to another shaft (such as nosecone shaft 158) and prosthetic valve 100 can move the capsule 160 over the length prosthetic valve 100 (i.e., when the prosthetic valve 100 is in the radially compressed configuration) such that the prosthetic valve 100 is enclosed within the capsule 160. In the case of a prosthetic valve 100^{b}, this can also keep the positioning arms 134 in a compacted state inside the capsule 160. Axial movement of the delivery shaft 154 in a proximal direction relative to another shaft (such as nosecone shaft 158) and prosthetic valve 100 can retract the capsule 160 from the prosthetic valve 100, exposing the prosthetic valve 100, for example, for deployment at an implantation location. Partial retraction of the capsule 160 can exposed a portion of the prosthetic valve 100 such that a portion of the frame 102 and/or other components of a prosthetic valve, such as positioning arms 134 in the case of prosthetic valve 100^{b}, can be expanded, while at least another portion of the frame 102 can remain in a compressed state. Knobs or other actuation mechanism at the handle of the delivery apparatus 152 can be used to control axial movement of the delivery shaft 154 and/or capsule 160.

In some examples, the delivery assembly 150 including the delivery apparatus 152 with the prosthetic valve 100, can be packaged in a sterile package that can be supplied to end users for storage and eventual use. In some examples, the leaflets 122 of the prosthetic valve 100 (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 100 and the delivery apparatus 152, respectively, can be free of any liquid. Methods for treating tissue leaflets for dry storage are disclosed in U.S. Pat. Nos. 8,007,992 and 8,357,387, both of which documents are incorporated herein by reference.

The prosthetic valve 100^{b} is illustrated to be at least partially retained in a crimped state within a capsule 160 in FIGS. 3A-3C. The capsule 160 of delivery apparatus 152^{a} can be either formed as a separate component attached to a distal end of the delivery shaft 154, or can be an integral end portion of the delivery shaft 154 in which at least a portion of the prosthetic valve 100^{b} can be similarly retained in a compressed state. In such cases, the term "capsule 160" can similarly refer to an integral distal portion of the delivery shaft 154, sized and shaped to retain at least a portion of the prosthetic valve 100 in a crimped or compressed state. In the case of a delivery assembly 150^{a} that includes a prosthetic valve 100^{b}, the capsule 160 can be sized to accommodate the compressed or crimped prosthetic valve 100^{b}, as well as the positioning arms 134 retained in a compacted state therein in a delivery configuration of the delivery assembly 150.

In the implantation procedure illustrated in FIGS. 3A-3D, the prosthetic valve 100^{b} is implanted in a native aortic valve 20 using a transfemoral delivery approach. In other examples, a prosthetic valve 100 can be implanted at other locations (e.g., a mitral valve, a tricuspid valve, and/or a pulmonary valve), within previously-implanted prosthetic valve, and/or using other delivery approaches (e.g., transapical, transaortic, transseptal, etc.).

As shown in FIG. 3A, the delivery assembly 150^{a}, including the delivery apparatus 152^{a} and the prosthetic valve 100^{b} retained in a compressed state, along with the positioning arms 134 compacted against the frame 102, is advanced through the patient's vasculature toward the site of implantation (e.g., the aortic annulus) over a guidewire 148. The distal end 162 of the capsule 160 can be retained in contact with, or in close proximity to, a rear portion of the nosecone 156, wherein the nosecone 156 has a tapering shape configured to assist in passage through the patient's vasculature. In some examples, as shown in FIG. 3A, the capsule 160 can be passed through the native leaflets 30 after entering the native aortic valve 20 through the aortic arch, positioning the capsule distal end 162 past the aortic annulus 24 and in the left ventricle 44, such as in the left ventricle outflow tract (LVOT).

As shown in FIG. 3B, the capsule 160 is then partially pulled in a proximal direction relative to the prosthetic valve 100^{b}, such as by partially pulling the delivery shaft 154, such that the positioning arms 134 are exposed and assume their deployed free state, wherein at least a portion of the frame 102 remains inside the capsule 160 at this stage. For example, the capsule 160 can be pulled in a manner that exposes at least a portion of the frame's inflow section 114, with the capsule distal end 162 positioned proximal to the free ends 138 of the arms 134 to fully expose the positioning arms 134, allowing them to assume their deployed free state, yet while keeping the capsule distal end 162 positioned distal to the outflow end 106 of the prosthetic valve 100, such that at least a portion of the frame 102, such as at least a portion of the outflow section 118, is retained in a compressed state inside the capsule 160.

In the example illustrated in FIGS. 3A-3B, the capsule 160 can be advanced sufficiently into the left ventricle 44 such that when the capsule 160 is partially retracted to expose the positioning arms 134, the arms 134 spring radially outwards into the sinuses 32 with the free ends 138 optionally positioned distal to the sinus ceilings 34, as illustrated in FIG. 3B. The axial position of the prosthetic valve 100^{b} can be then adjusted, such as by proximally pulling the prosthetic valve 100^{b}, optionally simultaneously with other components of the delivery apparatus 152, such as capsule 160 and/or delivery shaft 154, until the free ends 138 of the positioning arms 134 contact the sinus ceilings 34, as shown in FIG. 3C.

In some examples, a free end 138 of the positioning arm 134 is constructed to be atraumatic (e.g., blunt or otherwise lacking sharp edges) to avoid damage to the surrounding anatomy during operation. In some examples, the positioning arm 134 can have a contoured free end 138 having rounded tip, or a bent circular shape, oval shape (e.g., spoon- shaped), elliptical shape, C-shape, J-shape, or any other shape.

Using tactile feedback from the positioning arms 134 created by the contact of their free ends 138 with the sinus ceilings 34, the clinician can confirm the position of the STJ level, relative to which the prosthetic valve 100^{b} can be adequately positioned. Conventional techniques for prosthetic valve positioning inside the native annulus include injection of contrast media into the region of implantation to visualize anatomical structures which are otherwise invisible under fluoroscopy. Utilization of positioning arms 134 can provide an indication of the STJ level 28, relative to which the prosthetic valve 100^{b} can be maneuvered and positioned, without the need to introduce contrast media into the blood stream.

In some examples, the positioning arms 134 are visible under X-ray, for example by including or being formed of a radiopaque material, such that once contact with the sinus ceilings 34 is identified, indicative of a desired position of the prosthetic valve 100^{b} relative to the Valsalva sinuses 32 and the coronary ostia 36, 38, the prosthetic valve 100^{b} can be further deployed and implanted inside the native aortic valve 20. Thus, tactile or visual feedback from the positioning arms 134, as described herein, can provide an indication of contact with the sinus ceilings 34. In some examples, the positioning arms 134 are flexible enough to bend and/or otherwise change in shape when forcibly pressed against the sinus ceilings 34. In such examples, contact of the free ends 138 with the sinus ceilings 34 during pullback of the prosthetic valve 100^{b} can be identified by the change in shape of the positioning arms 134 and their free ends 138, visible for example under fluoroscopy, without the need to inject a contrast agent, such as Barium or other type of contrast agent, into the patient blood stream during the procedure, which can advantageously increase the safety of the procedure and reduce costs of materials.

It is to be understood that the clinician can rely on additional positioning techniques, such as fluoroscopy, echocardiography, etc. For example, during initial advancement of the delivery assembly 150^{a} into the heart, the clinician can use fluoroscopic, echocardiographic, and/or other imaging methods to provide visual confirmation of the orientation and position of the delivery assembly 150^{a}, including components thereof such as prosthetic valve 100^{b} and/or positioning arms 134, relative to the anatomical region of interest. The clinician can also use the fluoroscopic, echocardiographic, and/or other imaging methods to provide visual confirmation of the orientation and position of various components of the delivery assembly 150^{a} in addition to the tactile feedback provided by the positioning arms 134, e.g., during the positioning of the prosthetic valve 100^{b}. The tactile feedback thus provides the clinician with another important sensory cue to the relative position of the positioning arms and/or prosthetic valve with respect to the coronary ostia 36, 38.

In some examples, a positioning arm 134 can be relatively conspicuous under applicable image-guidance modalities, such as fluoroscopic, echocardiographic, and/or other imaging methods. As mentioned above, a positioning arm 134 can be made, in some examples, of a shape memory material such as Nitinol. As Nitinol is sometime hard to see on fluoroscopy, radiopaque markers (such as gold), could be added, or the Nitinol could be mixed with a radiopaque material for easy identification on fluoroscopy during the procedure. Thus, in some examples, the positioning arm 134 is either made of a radiopaque material or comprises one or more radiopaque markers. This can assist, as mentioned above, in identification of contact of the positioning arm 134 with the sinus ceiling 34. For example, when using fluoroscopy or other appropriate imaging technique, the clinician can see the proximal portion of the positioning arm 134, including free end 138, begin to bend, buckle, or otherwise deform in shape, when engaged with (i.e., pressed against) the sinus ceiling 34. Thus, contact of the positioning arm 134 with the sinus ceiling 34 can be identified by tactile feedback and/or visually from fluoroscopy or other image guidance.

When the capsule 160 is retracted to expose the positioning arms 134, it may be desirable to allow free undisturbed deployment of the positioning arms 134 to their free state, without being limited by anatomical structures that can surround a portion of the prosthetic valve 100^{b} during deployment. For example, part of the inflow section 114, which is a section of the frame 102 exposed along with the positioning arms 134 during partial retraction of the capsule 160, can reside inside the aortic annulus 24. Moreover, as part of the frame 102 is exposed and expanded inside the native aortic valve 20, a portion of the frame 102, such as at least part of the inflow section 114, can be surrounded by the native leaflets 30 which can be pushed against the prosthetic valve 100^{b} as illustrated in FIGS. 3B and 3C for example. In such cases, a movable part of the positioning arm 134 extending from the base 136 can be limited in movement if sandwiched between the frame 102 and any of the aortic annulus 24 or native leaflet 30. A prosthetic valve 100^{b} can be designed, in some examples, to avoid this by having the base 136 of the arm 134 sufficiently distanced from potential regions of interaction with the aortic annulus 24 or/or native leaflets 30. In some examples, the base 136 of a positioning arm 134 is proximal to the inflow end 104 of the prosthetic valve 100^{b}. In some examples, the base 136 is positioned at the inflow section 114 at a position that is proximally distanced from the inflow end 104. In some examples, the base 136 is positioned at the transition section 116.

In the example illustrated in FIGS. 2-3D, the bases 136 of the positioning arms 134 are proximal to the inflow end 104, while the free ends 138 are distal to the outflow end 106, and as mentioned above, are also proximal to the leaflets 122 of the prosthetic valve 100^{b}, such as by being proximal to their free edges 124.

The axial distance H_{D} between the free ends 138 of the arms 134 and the free edges 124 of the leaflets 122 (see FIGS. 2 and 3D), and/or other components of the prosthetic valve 100 that can limit flow or coronary access through the frame 102, such as commissures 128 and/or the outflow end of a skirt (such as any of an inner skirt 130 or a PVL skirt which is not shown in the illustrated example), is set to allow adequate passage of blood flow and/or passage of a coronary catheter through such a spaced gap. Thus, when the prosthetic valve 100^{b} is implanted in a native aortic valve 20 at a level in which the free ends 138 of the positioning arms 134 are engaged with the sinus ceilings 34, the axial distance H_{D} between the free ends 138 and the leaflets 122 of the valve 100^{b} ensures adequate perfusion therethrough into the coronary ostia 36, 38, and/or provides a sufficient gap G_{P} between the STJ level 28 and the free edges 124 of valvular structure 120 for passing a coronary catheter (such as a 6 French or any other appropriate catheter) into the coronary arteries 40, 42.

In some examples, the free ends 138 of the arms 134 are axially distanced from the free edges 124 of the leaflets 122 in a free state of the positioning arms 134, such that a coronary catheter having an outer diameter of 6 Fr. can pass through the frame 102 within this distance. In some examples, the axial distance between the free ends 138 of the arms 134 and the free edges 124 of the leaflets 122, is equal to or greater than 2 millimeters (mm.) in a free state of the positioning arms 134. The leaflets 122 of some types of prosthetic valves 100 can be inclined towards the center of the valve 100, in which case, a reference to an axial distance of the free ends 138 from the free edges 124 refer to a portion of the free edges 124 at or in close proximity to the frame 102.

As shown in FIG. 3D, once proper axial positioning of the prosthetic valve 100^{b} and its positioning arms 134 is achieved, the capsule 160 can be further pulled, relative to the prosthetic valve 100^{b}, to fully expose the remaining outflow section 118, allowing it to expand and anchor against the ascending aorta 26. The delivery apparatus can be then released from the prosthetic valve 100^{b}, such as by releasing retaining structures of the delivery apparatus 152 from retaining elements 132 of the frame 102, after which the delivery apparatus 152 can be withdrawn from the patient, leaving the prosthetic valve 100^{b} implanted in the native aortic valve 20.

While FIGS. 3A-3D are illustrated and described for use with a self-expandable prosthetic valve 100^{b}, in some examples, the prosthetic valve 100 can include other types of frames, such as a balloon expandable frame or a mechanically expandable frame, while the positioning arms 134 are similarly configured to self-expand away from the frame in their free state. For example, a balloon expandable prosthetic valve can be mounted on an inflatable balloon at a distal end of a balloon catheter, and delivered inside a capsule 160 disposed around the prosthetic valve towards and into the native aortic valve 20, in a similar manner to that described above with respect to FIG. 3A. The capsule can be then retracted to expose the positioning arms 134, in a similar manner to that described above with respect to FIG. 3B, with the exception that the capsule can be fully retracted to expose the entire prosthetic valve, as there is no risk of the valve fully expanding at this stage as loon as the balloon remains deflated. After the prosthetic valve and balloon catheter are axially maneuvered up to contact of the free ends 138 of the arms 134 with the sinus ceilings 34, the balloon can be inflated to expand the prosthetic valve, after which the balloon can be deflated and the delivery apparatus can be retrieved from the patient's body.

In the case of a prosthetic valve having a mechanically expandable frame, the steps of delivering the delivery assembly to the site of implantation, and exposing the positioning arms 134 by pulling the capsule, in a manner that can optionally expose the entire prosthetic valve, can be performed in a similar manner, and the axial position of the prosthetic valve can be adjusted along with actuation drives that can be releasably coupled thereto. After an adequate axial position is achieved, the frame can be expanded, for example by actuation drivers of the delivery apparatus coupled to the frame, after which the drivers can be released from the prosthetic valve and the delivery apparatus can be similarly retrieved from the patient's body.

FIGS. 4A-4D show a distal portion of an exemplary delivery assembly 150^{c} used in a method for implantation of a prosthetic valve 100 within a native aortic valve 20. Delivery assembly 150^{c} is an exemplary implementation of delivery assembly 150, and thus can include any of the features described above for delivery assembly 150, except that the delivery apparatus 152^{c} of delivery assembly 150^{c} includes two capsules, namely a first capsule 170 and a second capsule 178 which are axially movable relative to each other, and one or more positioning arms 190 attached to the first capsule 170. A delivery apparatus 152^{c} equipped with positioning arms 190 can be used for delivery and implantation of a prosthetic valve 100 that does not necessarily include its own positioning arms, such as the prosthetic valve 100^{a} shown in FIG. 1.

The first capsule 170 can be attached to a distal end of the delivery shaft 154, configured to retain at least a portion of the prosthetic valve 100 in a compressed state therein. The second capsule 178 can be axially separated from the first capsule 170, and is similarly configured to retain at least a portion of the prosthetic valve 100 in a compressed state therein, which can be a different portion of the valve 100 than that retained inside of the first capsule 170. The first capsule 170 extends towards a first capsule distal end portion 172 terminating with a first capsule distal edge 174, and defines a first capsule outer surface 176. The second capsule 178 extends between a second capsule proximal edge 182 and a second capsule distal end 186, and comprises a second capsule proximal portion 180 extending distally from the second capsule proximal edge 182, and a second capsule distal portion 184 extending from the second capsule proximal portion 180 to the second capsule distal end 186. The second capsule 178 defines a second capsule inner surface 188.

First capsule 170 can be either formed as a separate component attached to a distal end of the delivery shaft 154, or can be an integral end portion of the delivery shaft 154 in which at least a portion of the prosthetic valve 100 can be retained in a compressed state. Second capsule 178 can be directly or indirectly coupled to a different shaft of the delivery apparatus 152^{a}, axially movable relative to the delivery shaft 154. In some examples, second capsule 178 can be coupled directly or indirectly to the nosecone shaft 158. In some examples, second capsule 178 can be coupled nosecone 156. In some examples, second capsule 178 can be either formed as a separate component attached to a proximal end of the nosecone 156, or can be an integral part of the nosecone 156, extending proximally from a tapering portion of the nosecone 156.

As shown for example in FIGS. 4B-4C, the delivery apparatus 152^{c} comprises one or more positioning arms 190 configured to assist in axial positioning during implantation inside the native aortic valve 20. While two positioning arms 190 are illustrated, it is to be understood that any other number is contemplated, including a single positioning arm, three positioning arms (for example, for placement in each of the three Valsalva sinuses 32), or more than three positioning arms. Any reference to a "positioning arm 190" in a singular form may refer to any plurality of positioning arms 190, and any reference to "positioning arms 190" in the plural form may similarly refer to a single positioning arm 190, unless stated otherwise.

The positioning arms 190 can be self-expandable arms that can be deployed into the sinuses 32. A positioning arm 190 extends from a base 192 at which it is attached to the first capsule 170, to a free end 194 which is biased radially away from the first capsule 170 to a position proximal to the base 192 and spaced away from the first capsule outer surface 176 in a free state of the positioning arm 190, referring to a state in which the positioning arm 190 is free to assume a pre-shaped configuration in the absence of external forces acting there-against.

The positioning arms 190 are affixed to the first capsule 170, such as to the first capsule distal end portion 172, at their bases 136, optionally by welding, gluing, embedding, overmolding, and the like. When a plurality of positioning arms 190 are provided, they can be equally or unequally spaced from each other around the circumference of the first capsule 170.

The positioning arms 190 can transition between a compacted state, in which the arms 190 can be pressed against the first capsule outer surface 176 and optionally assume a relatively elongated configuration that can be parallel to a longitudinal central axis of the first capsule 170, wherein the free ends 194 contact or are in close proximity to the first capsule outer surface 176 as shown for example in FIG. 4D, and a deployed or free state in which the arms 190 are allowed to extend away from the first capsule 170 such that the free ends 194 of the arms 190 are radially spaced from the first capsule 170, as shown for example in FIG. 4B-4C. In some examples, the positioning arms 190 can be made of a shape memory material, such as a nickel-titanium alloy (for example, Nitinol), and are preset to a shape of the free state by heat treatment. The positioning arms 190 can be configured to assume a compacted state when constrained by a radial constrain of a delivery apparatus and/or when pulled by tensioning members 196 or equivalent tensioning members that may be attached thereto, and to be released to assume their free state after being released from the radial constrain while the tensioning members 196 are untensioned, which can be performed prior to full deployment of the prosthetic valve 100.

When the second capsule 178 is coupled to the nosecone shaft 158 and/or the nosecone 156, axial movement of the nosecone shaft 158 relative to the delivery shaft 154 in the proximal or distal directions will cause similar axial movement of the second capsule 178 proximally towards the first capsule 170 or distally away from the first capsule 170.

During delivery to the site of implantation, the prosthetic valve 100 can be retained in a crimped state inside the first and second capsules 170, 178, such as by having at least part of the outflow section 118 compressed inside the first capsule 170, and at least part of the inflow section 114 compressed inside the second capsule 178.

The positioning arms 190 proximally extend from the bases 192. When the prosthetic valve 100 is at least partially retained in a compressed state in the first capsule 170, the axial position of the outflow section 118 inside the first capsule 170 can be selected, for example relative to the first capsule distal edge 174, such that the free ends 194 of the capsule's positioning arms 190 are positioned proximal to the leaflets 122 of the valvular structure 120 in a free state of the arms 190. In some examples, the free ends 194 are proximal to the free edges 124 of the leaflets 122 in a free state of the arms 134, when the prosthetic valve 100 is loaded into the first capsule 170. In some examples, the free ends 194 are proximal to the commissures 128, when the prosthetic valve 100 is loaded into the first capsule 170. In some examples, the free ends 194 are proximal to the skirt 130 in a free state of the arms 190, when the prosthetic valve 100 is loaded into the first capsule 170. The positioning arms 190 can be transitioned into a compacted state prior to, during or after loading the prosthetic valve 100 into the first capsule 170.

The positioning arms 190 are configured to engage with the sinuses 32 such that the free ends 194 can contact the sinus ceilings 34 when the arms 190 are deployed in the aortic valve 20. As mentioned above, a prosthetic valve 100 can include an outflow section 118 configured to extend into and engage with the ascending aorta 26, above the STJ level 28, when fully deployed out of the first and second capsules 170, 178. The free ends 194 of the arms 190 extending from the first capsule 170 are configured to contact the sinus ceilings 34 which are substantially at, or in close proximity to, the STJ level 28, at a position in which a subsequent full deployment of the prosthetic valve 100 will retain a minimal gap G_{P} axially measured between the STJ level 28 and the outflow end 106 of the frame 102.

In the implantation procedure illustrated in FIGS. 4A-4E, delivery assembly 150^{c} is utilized to implant the prosthetic valve 100 in a native aortic valve 20 using a transfemoral delivery approach. In other examples, a delivery assembly 150^{c} can be used to implant a prosthetic valve 100 at other locations (e.g., a mitral valve, a tricuspid valve, and/or a pulmonary valve), within previously-implanted prosthetic valve, and/or using other delivery approaches (e.g., transapical, transaortic, transseptal, etc.).

As shown in FIG. 4A, the delivery assembly 150^{c}, including the delivery apparatus 152^{c} and the prosthetic valve 100 such as valve 100^{a} retained in a compressed state inside the first and second capsules 170, 178, while the positioning arms 190 are compacted against the first capsule 170, is advanced through the patient's vasculature toward the site of implantation (e.g., the aortic annulus) over a guidewire 148. In this delivery configuration, when the prosthetic valve 100 is compressed inside the first and second capsules 170 and 178, the second capsule proximal portion 180 covers at least part of the first capsule 170 extending from the first capsule distal edge 174, such that the positioning arms 190 are kept in a compacted state between the first capsule 170 and the second capsule proximal portion 180. The positioning arms 190 can be retained in a compacted state, as shown in FIG. 4A, between the first capsule outer surface 176 and the second capsule inner surface 188 during advancement through the patient's vasculature.

The second capsule distal end 186, which is distal to the first capsule distal edge 174, can be retained in contact with, or in close proximity to, a rear portion of the nosecone 156. The second capsule distal portion 184 can extend distally from the first capsule distal edge 174 when the second capsule proximal portion 180 is disposed around the positioning arms 190 in the delivery configuration. In some examples, the inner diameter of the second capsule proximal portion 180 is greater than the inner diameter of the second capsule distal portion 184. The inner diameter of the first capsule 170 can be designed to accommodate an appropriate portion of the prosthetic valve 100 in a compressed state therein, such as at least part of a compressed outflow section 118. The inner diameter of the second capsule distal portion 184 can be designed to accommodate an appropriate portion of the prosthetic valve 100 in a compressed state therein, such as at least part of a compressed inflow section 114. The inner diameter of the second capsule proximal portion 180 can be designed to accommodate both the first capsule 170 and the positioning arms 190 extending therefrom, in a compacted state of the arms 190. Thus, the second capsule proximal portion 180 can serve as an outer restraining component configured to force the positioning arms 190 to assume their compacted state between both capsules 170 and 178.

In some examples, as further shown in FIG. 4A, the second capsule 178 can be passed through the native leaflets 30 after entering the native aortic valve 20 through the aortic arch, positioning the second capsule distal end 186 past the aortic annulus 24 and in the left ventricle 44, such as in the left ventricle outflow tract (LVOT).

As shown in FIG. 4B, axial movement of one of the capsules 170, 178 relative to the other can uncover and expose the positioning arms 190, allowing them to assume their deployed free state, wherein at least a portion of the frame 102 remains inside the first capsule 170 and optionally with the second capsule 178 as well at this stage. Uncovering the positioning arms 190 is achieved by axially moving at least one of the capsules relative to the other such that the second capsule proximal edge 182 is distal to the bases 192 of the arms 190, and is optionally distal to the first capsule distal end portion 172 and/or first capsule distal edge 174. This can be accomplished by moving the first capsule 170 proximally relative to the second capsule 178, by moving the second capsule 178 distally relative to the first capsule 170, or both, while the prosthetic valve 100 remains axially immovable relative to the first capsule 170.

In the example illustrated in FIGS. 4A-4B, the second capsule 178 is advanced into the left ventricle 44 while a sufficient portion of the first capsule 170 remains above the aortic annulus 24, such that when the first capsule 170 is uncovered from the second capsule 178 to expose the positioning arms 190, the arms 190 spring radially outwards into the sinuses 32 with the free ends 194 optionally positioned distal to the sinus ceilings 34, as illustrated in FIG. 4B. The axial position of the prosthetic valve 100 can be then adjusted, such as by proximally pulling the prosthetic valve 100 simultaneously with the first capsule 170, until the free ends 194 of the positioning arms 190 contact the sinus ceilings 34, as shown in FIG. 4C.

The second capsule 178 can be further pushed in a distal direction relative to the prosthetic valve 100 and/or the first capsule 170, such as by partially pushing the nosecone shaft 158 or any other shaft to which the second capsule 178 is coupled, in a manner that exposes and releases at least a portion of the frame's inflow section 114, from the second capsule 178, while at least part of the outflow section 118 remains in a compressed state inside the first capsule 170, as also shown in FIG. 4C.

It is to be understood that exposing the prosthetic valve 100 from the second capsule 178 can be performed prior to, simultaneously with, or subsequent to, axial position adjustment of the prosthetic valve 100 with the first capsule 170 to a position of the free ends 194 of the positioning arms 190 pushed against the sinus ceilings 34.

In some examples, a free end 194 of the positioning arm 190 is constructed to be atraumatic (e.g., blunt or otherwise lacking sharp edges) to avoid damage to the surrounding anatomy during operation. In some examples, the positioning arm 190 can have a contoured free end 194 having rounded tip, or a bent circular shape, oval shape (e.g., spoon- shaped), elliptical shape, C-shape, J-shape, or any other shape.

Using tactile feedback from the positioning arms 190 created by the contact of their free ends 194 with the sinus ceilings 34, the clinician can confirm the position of the STJ level, relative to which the prosthetic valve 100 can be adequately positioned. Conventional techniques for prosthetic valve positioning inside the native annulus include injection of contrast media into the region of implantation to visualize anatomical structures which are otherwise invisible under fluoroscopy. Utilization of positioning arms 190 can provide an indication of the STJ level 28, relative to which the prosthetic valve 100 can be maneuvered and positioned, without the need to introduce contrast media into the blood stream.

In some examples, the positioning arms 190 are visible under X-ray, for example by including or being formed of a radiopaque material, such that once contact with the sinus ceilings 34 is identified, indicative of a desired position of the prosthetic valve 100 relative to the Valsalva sinuses 32 and the coronary ostia 36, 38, the prosthetic valve 100 can be further deployed and implanted inside the native aortic valve 20. Thus, tactile or visual feedback from the positioning arms 190, as described herein, can provide an indication of contact with the sinus ceilings 34. In some examples, the positioning arms 190 are flexible enough to bend and/or otherwise change in shape when forcibly pressed against the sinus ceilings 34. In such examples, contact of the free ends 194 with the sinus ceilings 34 during pullback of the first capsule 170 (along with the prosthetic valve 100) can be identified by the change in shape of the positioning arms 190 and their free ends 194, visible for example under fluoroscopy, without the need to inject a contrast agent, such as Barium or other type of contrast agent, into the patient blood stream during the procedure, which can advantageously increase the safety of the procedure and reduce costs of materials.

It is to be understood that the clinician can rely on additional positioning techniques, such as fluoroscopy, echocardiography, etc. For example, during initial advancement of the delivery assembly 150^{c} into the heart, the clinician can use fluoroscopic, echocardiographic, and/or other imaging methods to provide visual confirmation of the orientation and position of the delivery assembly 150^{c}, including components thereof such as prosthetic valve 100^{a} and/or positioning arms 190, relative to the anatomical region of interest. The clinician can also use the fluoroscopic, echocardiographic, and/or other imaging methods to provide visual confirmation of the orientation and position of various components of the delivery assembly 150^{a} in addition to the tactile feedback provided by the positioning arms 190, e.g., during the positioning of the prosthetic valve 100^{a}. The tactile feedback thus provides the clinician with another important sensory cue to the relative position of the positioning arms and/or prosthetic valve with respect to the coronary ostia 36, 38.

In some examples, a positioning arm 190 can be relatively conspicuous under applicable image-guidance modalities, such as fluoroscopic, echocardiographic, and/or other imaging methods. As mentioned above, a positioning arm 190 can be made, in some examples, of a shape memory material such as Nitinol. As Nitinol is sometime hard to see on fluoroscopy, radiopaque markers (such as gold), could be added, or the Nitinol could be mixed with a radiopaque material for easy identification on fluoroscopy during the procedure. Thus, in some examples, the positioning arm 190 is either made of a radiopaque material or comprises one or more radiopaque markers. This can assist, as mentioned above, in identification of contact of the positioning arm 190 with the sinus ceiling 34. For example, when using fluoroscopy or other appropriate imaging technique, the clinician can see the proximal portion of the positioning arm 190, including free end 194, begin to bend, buckle, or otherwise deform in shape, when engaged with (i.e., pressed against) the sinus ceiling 34. Thus, contact of the positioning arm 190 with the sinus ceiling 34 can be identified by tactile feedback and/or visually from fluoroscopy or other image guidance.

The axial position of the free ends 194 of the arms 190 in the free state, relative to the free edges 124 of the leaflets 122 in a theoretical expanded state of the frame 102, but while the prosthetic valve 100 is loaded inside the first capsule 170, is set to allow adequate passage of blood flow and/or passage of a coronary catheter through such a spaced gap. While described with respect to the distance from the free edges 124 of the leaflets 122, this can be similarly measured relative to other components of the prosthetic valve 100 that can limit flow or coronary access through the frame 102, such as commissures 128 and/or the outflow end of a skirt (such as any of an inner skirt 130 or a PVL skirt which is not shown in the illustrated example).

When the first capsule 170 is uncovered from the second capsule 178 to expose the positioning arms 190, it may be desirable to allow free undisturbed deployment of the positioning arms 190 to their free state, without being limited by anatomical structures that can surround a portion of the first capsule 170 during the procedure. For example, a movable part of the positioning arm 190 extending from the base 192 can be limited in movement if sandwiched between the first capsule 170 and any of the aortic annulus 24 or native leaflet 30. Relative positioning of the prosthetic valve 100 in a compressed state inside the first capsule 170 prior to insertion into the patient's body can be set to avoid this by having the base 192 of the arm 190 at the first capsule 170 sufficiently distanced from potential regions of interaction with the aortic annulus 24 or/or native leaflets 30 during positioning of the arms 190 inside the native valve 20. In some examples, the prosthetic valve 100 is loaded into the first capsule 170 prior to insertion into the patient's body, such that the base 192 of a positioning arm 190 at the first capsule 170 is proximal to the inflow end 104 of the prosthetic valve 100. In some examples, the prosthetic valve 100 is loaded into the first capsule 170 prior to insertion into the patient's body, such that the base 192 is positioned at a level of the inflow section 114 that is proximally distanced from the inflow end 104. In some examples, the prosthetic valve 100 is loaded into the first capsule 170 prior to insertion into the patient's body, such that the base 136 is positioned at a level of the transition section 116.

As shown in FIG. 4D, once proper axial positioning of the prosthetic valve 100 and the positioning arms 190 extending from the first capsule 170 is achieved, the positioning arms 190 can be transitioned back to the compacted state. Is some examples, tensioning members 196 or tethers can be coupled to the positioning arms 190. For example, distal ends of the tensioning members 196 can be attached, such as by being tied, glued, or otherwise coupled, to the free ends 194 of the positioning arms 190. The tensioning members 196 or tethers can be in the form of pull wires, cables, strings, sutures, and the like. The number of tensioning members 196 can match the number of positioning arms 190. The proximal ends of the tensioning members 196 (not shown) can extend to the handle of the delivery apparatus, and be operated by a knob to apply tension thereto or release tension therefrom. When the tensioning members 196 are tensioned, as shown in FIG. 4D, the positioning arms 190 can be forced to assume their compacted state, substantially parallel to a central longitudinal axis of the first capsule 170.

In some examples, the first capsule 170 and/or the delivery shaft 154 can include one or more side openings 198 which can be formed at or proximal to the axial position of the free ends 194, optionally in the free state of the arms 190. The number of side openings 198 can match the number of tensioning members 196, such that each tensioning member can extend along a lumen of the delivery shaft 154 and/or the first capsule 170, through a corresponding side opening 198, to a connection with the free end 194 of the corresponding positioning arm 190.

As long as no tension is applied to the positioning arms 190 by the tensioning members 196, the positioning arms 190 can spring radially away from first capsule 170 (assuming that the arms 190 are not covered by the second capsule 178), such that the free ends 194 are positioned radially farther away from the first capsule 170, and can be generally laterally and proximally-oriented in the free state as shown in FIGS. 4B and 4C.

In order to transition the positioning arms 190 back to a compacted state, prior to full deployment of the prosthetic valve 100 from the first capsule 170, tension can be applied to the tensioning members 196 to force the positioning arms 190 to re-assume the compacted state as shown in FIG. 4D.

As further shown in FIG. 4E, the first capsule 170 can be then proximally pulled relative to the prosthetic valve 100, to fully expose the remaining outflow section 118, allowing it to expand and anchor against the ascending aorta 26. The delivery apparatus can be then released from the prosthetic valve 100, such as by releasing retaining structures of the delivery apparatus 152^{a} from retaining elements 132 of the frame 102, after which the delivery apparatus 152^{a} can be withdrawn from the patient, leaving the prosthetic valve 100 implanted in the native aortic valve 20.

This leaves the prosthetic valve 100 implanted in a native aortic valve 20 is a position that ensures adequate perfusion through the valve 100 into the coronary ostia 36, 38, and/or provides a sufficient gap G_{P} between the STJ level 28 and the free edges 124 of valvular structure 120 for passing a coronary catheter (such as a 6 French or any other appropriate catheter) into the coronary arteries 40, 42.

Any of the delivery assemblies or prosthetic valve disclosed herein can be sterilized (for example, with heat, radiation, and/or chemicals, etc.) to ensure they are safe for use with patients, and any of the methods herein can include sterilization of the associated delivery assembly or prosthetic valve as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Technology

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising:

a frame movable between a radially compressed state and a radially expanded state;

a valvular structure mounted within the frame and comprising a plurality of leaflets configured to regulate flow through the prosthetic valve; and

at least one positioning arm extending proximally from a base affixed to the frame, to a free end, wherein the at least one positioning arm is configured to transition between a compacted state and a free state;

wherein the at least one positioning arm is biased to the free state in which the free end is radially spaced from the frame and is distal to a proximal end of the frame.

Example 2. The prosthetic valve of any example herein, particularly of example 1, wherein the base is proximal to a distal end of the frame.

Example 3. The prosthetic valve of any example herein, particularly of example 1 or 2, wherein the free end is proximal to the valvular structure in the free state.

Example 4. The prosthetic valve of any example herein, particularly of example 3, wherein each leaflet comprises a free edge, and wherein the free end is proximal to free edges of the leaflets.

Example 5. The prosthetic valve of any example herein, particularly of example 4, wherein the axial distance between the free edge of the at least one positioning member and the free edges of the leaflets is greater than 2 mm.

Example 6. The prosthetic valve of any example herein, particularly of example 3, wherein the valvular structure comprises commissures coupled to commissure attachment features of the frame, and wherein the free end is proximal to commissures in the free state.

Example 7. The prosthetic valve of any example herein, particularly of example 6, wherein the axial distance between the free end of the at least one positioning member and the commissures is greater than 2 mm.

Example 8. The prosthetic valve of any example herein, particularly of any one of examples 1 to 7, further comprising an inner skirt disposed around the frame.

Example 9. The prosthetic valve of any example herein, particularly of example 8, wherein the leaflets are coupled to the inner skirt along a scalloped line.

Example 10. The prosthetic valve of any example herein, particularly of example 8 or 9, wherein the free end is proximal to the inner skirt in the free state.

Example 11. The prosthetic valve of any example herein, particularly of example 10, wherein the axial distance between the free end of the at least one positioning member and the inner skirt is greater than 2 mm.

Example 12. The prosthetic valve of any example herein, particularly of any one of examples 1 to 11, wherein the frame comprises an inflow section and an outflow section having a diameter greater than that of the inflow section in the expanded state.

Example 13. The prosthetic valve of any example herein, particularly of example 12, wherein the inflow section is configured to anchor the prosthetic valve against a native annulus when The prosthetic valve of any example herein, particularly is implanted in a native heart valve.

Example 14. The prosthetic valve of any example herein, particularly of example 13, wherein the outflow section is configured to anchor the prosthetic valve against the ascending aorta when the prosthetic valve is implanted in the native heart valve.

Example 15. The prosthetic valve of any example herein, particularly of any one of examples 12 to 14, wherein the base is affixed to the inflow section.

Example 16. The prosthetic valve of any example herein, particularly of any one of examples 12 to 14, wherein the frame further comprises a transition section between the inflow section and the outflow section, and wherein the base is affixed to the transition section.

Example 17. The prosthetic valve of any example herein, particularly of any one of examples 1 to 16, wherein the free end is atraumatic.

Example 18. The prosthetic valve of any example herein, particularly of any one of examples 1 to 17, wherein the free end is configured to engage with a sinus ceiling of a Valsalva sinus.

Example 19. The prosthetic valve of any example herein, particularly of example 18, wherein, when the free end is engaged with the sinus ceiling, an axial gap is defined between an STJ level and the valvular structure.

Example 20. The prosthetic valve of any example herein, particularly of example 19, wherein the axial gap is greater than 2 mm.

Example 21. The prosthetic valve of any example herein, particularly of any one of examples 1 to 20, wherein the at least one positioning arm is made from a shape-memory material.

Example 22. The prosthetic valve of any example herein, particularly of example 21, wherein the shape-memory material is Nitinol.

Example 23. The prosthetic valve of any example herein, particularly of any one of examples 1 to 22, wherein the at least one positioning arm comprises a plurality of positioning arms.

Example 24. The prosthetic valve of any example herein, particularly of any one of examples 1 to 23, wherein the at least one positioning arm comprises a radiopaque material.

Example 25. The prosthetic valve of any example herein, particularly of any one of examples 1 to 24, wherein the plurality of leaflets comprises three leaflets.

Example 26. The prosthetic valve of any example herein, particularly of any one of examples 1 to 25, wherein the frame is self-expandable.

Example 27. A method of implanting a prosthetic valve, comprising:

advancing a delivery assembly that comprises a delivery apparatus carrying a prosthetic valve retained in a radially compressed state inside a capsule of the delivery apparatus, to a native heart valve;

exposing at least a portion of an inflow section of the prosthetic valve and one or more positioning arms affixed to a frame of the prosthetic valve, such that free ends of the positioning arms spring outwardly towards Valsalva sinuses of the native heart valve, while at least a portion of an outflow section of the prosthetic valve remains retained inside the capsule;

adjusting the axial position of the prosthetic valve such that the free ends of the positioning arms engage with sinus ceilings of the Valsalva sinuses; and

exposing the remainder of the prosthetic valve from the capsule.

Example 28. The method of any example herein, particularly of example 27, wherein the frame is movable between a radially compressed state and a radially expanded state.

Example 29. The method of any example herein, particularly of example 28, wherein the frame is self-expandable.

Example 30. The method of any example herein, particularly of any one of examples 27 to 29, wherein the advancing the delivery assembly comprises retaining the positioning arms in a compacted state inside the capsule.

Example 31. The method of any example herein, particularly of any one of examples 27 to 30, wherein each of the one or more positioning arms extends proximally from a base at which it is affixed to the frame, to the free end.

Example 32. The method of any example herein, particularly of example 31, wherein the base is proximal to a distal end of the frame.

Example 33. The method of any example herein, particularly of example 31 or 32, wherein the base is affixed to the inflow section.

Example 34. The method of any example herein, particularly of any one of examples 31 to 33, wherein the adjusting the axial position comprises positioning the bases of the one or more positioning arms proximal to a native annulus of the native heart valve.

Example 35. The method of any example herein, particularly of any one of examples 31 to 34, wherein the adjusting the axial position comprises positioning the bases of the one or more positioning arms proximal to native leaflets of the native heart valve.

Example 36. The method of any example herein, particularly of any one of examples 27 to 35, wherein the adjusting the axial position comprises pressing the free ends against the sinus ceilings.

Example 37. The method of any example herein, particularly of any one of examples 27 to 36, wherein the prosthetic valve further comprises a valvular structure mounted within the frame, the valvular structure comprising a plurality of leaflets configured to regulate flow through the prosthetic valve.

Example 38. The method of any example herein, particularly of example 37, wherein, when the free ends are engaged with the sinus ceilings, an axial distance of at least 2 mm. is defined between the free ends and the valvular structure.

Example 39. The method of any example herein, particularly of example 37 or 38, wherein, when the free ends are engaged with the sinus ceilings, an axial distance of at least 2 mm. is defined between an STJ level of the native heart valve and the valvular structure.

Example 40. The method of any example herein, particularly of example 37, wherein, when the free ends are engaged with the sinus ceilings, an axial distance of at least 2 mm. is defined between the free ends of the positioning arms and free edges of the leaflets.

Example 41. The method of any example herein, particularly of example 37, wherein, when the free ends are engaged with the sinus ceilings, an axial distance of at least 2 mm. is defined between an STJ level of the native heart valve and free edges of the leaflets.

Example 42. The method of any example herein, particularly of example 37, wherein, when the free ends are engaged with the sinus ceilings, an axial distance of at least 2 mm. is defined between the free ends and commissures of the valvular structure.

Example 43. The method of any example herein, particularly of example 37, wherein, when the free ends are engaged with the sinus ceilings, an axial distance of at least 2 mm. is defined an STJ level of the native heart valve and commissures of the valvular structure.

Example 44. The method of any example herein, particularly of example 37, wherein, when the free ends are engaged with the sinus ceilings, an axial distance of at least 2 mm. is defined between the free ends and an inner skirt of the prosthetic valve.

Example 45. The method of any example herein, particularly of example 37, wherein, when the free ends are engaged with the sinus ceilings, an axial distance of at least 2 mm. is defined between an STJ level of the native heart valve and an inner skirt of the prosthetic valve.

Example 46. The method of any example herein, particularly of any one of examples 27 to 45, wherein the free ends are atraumatic.

Example 47. The method of any example herein, particularly of any one of examples 27 to 46, wherein the exposing the remainder of the prosthetic valve comprises allowing the prosthetic valve to expand against the native heart valve.

Example 48. The method of any example herein, particularly of any one of examples 27 to 47, wherein the exposing the remainder of the prosthetic valve comprises anchoring the inflow section against a native annulus of the native heart valve.

Example 49. The method of any example herein, particularly of any one of examples 27 to 48, wherein the exposing the remainder of the prosthetic valve comprises anchoring the outflow section against an ascending aorta proximal to the Valsalva sinuses.

Example 50. The method of any example herein, particularly of any one of examples 27 to 49, wherein the free ends are distal to a proximal end of the frame.

Example 51. The method of any example herein, particularly of any one of examples 27 to 50, wherein the one or more positioning arms are made from a shape-memory material.

Example 52. The method of any example herein, particularly of example 51, wherein the shape-memory material is Nitinol.

Example 53. The method of any example herein, particularly of any one of examples 27 to 52, wherein the adjusting the axial position comprises using tactile feedback from the one or more positioning members to confirm engagement with the sinus ceilings.

Example 54. The method of any example herein, particularly of any one of examples 27 to 53, wherein the one or more positioning members is radiopaque, and wherein the adjusting the axial position comprises identifying, under image guidance, deformation of the one or more positioning members due to engagement with the sinus ceilings.

Example 55. The method of any example herein, particularly of any one of examples 27 to 54, further comprising, subsequent to the exposing, releasing the delivery apparatus from the prosthetic valve.

Example 56. The method of any example herein, particularly of example 55, wherein the releasing comprises decoupling the delivery apparatus from retaining elements of the prosthetic valve.

Example 57. The method of any example herein, particularly of example 55 or 56, further comprising, subsequent to the releasing, withdrawing the delivery apparatus from the native heart valve.

Example 58. The method of any example herein, particularly of any one of examples 27 to 57, wherein the native heart valve is a native aortic valve.

Example 59. A delivery assembly comprising:

a prosthetic valve comprising a frame movable between a radially compressed state and a radially expanded state, and a valvular structure mounted within the frame and comprising a plurality of leaflets configured to regulate flow through the prosthetic valve; and

a delivery apparatus comprising:

a delivery shaft;

a first capsule at a distal end of the delivery shaft;

a second capsule axially movable relative to the first capsule; and

at least one positioning arm extending proximally from a base affixed to the first capsule, to a free end, wherein the at least one positioning arm is configured to transition between a compacted state and a free state;

wherein the at least one positioning arm is biased to the free state in which the free end is radially spaced from the first capsule.

Example 60. The delivery assembly of any example herein, particularly of example 59, wherein the frame comprises an inflow section and an outflow section having a diameter greater than that of the inflow section in the expanded state.

Example 61. The delivery assembly of any example herein, particularly of example 60, wherein the second capsule is configured to retain therein at least a portion of the inflow section in the compressed state.

Example 62. The delivery assembly of any example herein, particularly of example 60 or 61, wherein the first capsule is configured to retain therein at least a portion of the outflow section in the compressed state.

Example 63. The delivery assembly of any example herein, particularly of example 62, wherein the free end is proximal to a proximal end of the frame, in the free state of the positioning arm, when the outflow portion is retained inside the first capsule.

Example 64. The delivery assembly of any example herein, particularly of example 62 or 63, wherein the free end is distal to valvular structure, in the free state of the positioning arm, when the outflow portion is retained inside the first capsule.

Example 65. The delivery assembly of any example herein, particularly of any one of examples 62 to 64, wherein the free end is distal to free edges of the leaflets, in the free state of the positioning arm, when the outflow portion is retained inside the first capsule.

Example 66. The delivery assembly of any example herein, particularly of any one of examples 62 to 65, wherein the free end is distal to commissures of the valvular structure, in the free state of the positioning arm, when the outflow portion is retained inside the first capsule.

Example 67. The delivery assembly of any example herein, particularly of any one of examples 62 to 66, wherein the free end is distal to an inner skirt of the prosthetic valve, in the free state of the positioning arm, when the outflow portion is retained inside the first capsule.

Example 68. The delivery assembly of any example herein, particularly of any one of examples 59 to 67, wherein the base is affixed to a distal end portion of the first capsule.

Example 69. The delivery assembly of any example herein, particularly of any one of examples 59 to 68, wherein the second capsule comprises a second capsule proximal portion and a second capsule distal portion, and wherein the second capsule proximal portion is configured to cover at least part of the first capsule.

Example 70. The delivery assembly of any example herein, particularly of example 69, wherein an inner diameter defined by the second capsule proximal portion is greater than an inner diameter defined by the second capsule distal portion.

Example 71. The delivery assembly of any example herein, particularly of example 69 or 70, wherein the second capsule proximal portion is configured to cover at least part of the at least one positioning arm.

Example 72. The delivery assembly of any example herein, particularly of example 71, wherein the second capsule proximal portion is configured to maintain the at least one positioning arm in the compacted state when the second capsule proximal portion covers the positioning arm.

Example 73. The delivery assembly of any example herein, particularly of example 72, wherein the at least one positioning arm is configured to spring outwards to the free state when uncovered from the second capsule.

Example 74. The delivery assembly of any example herein, particularly of any one of examples 59 to 73, wherein the free end is atraumatic.

Example 75. The delivery assembly of any example herein, particularly of any one of examples 59 to 74, wherein the free end is configured to engage with a sinus ceiling of a Valsalva sinus.

Example 76. The delivery assembly of any example herein, particularly of any one of examples 59 to 75, wherein the frame is self-expandable.

Example 77. The delivery assembly of any example herein, particularly of any one of examples 59 to 75, wherein the at least one positioning arm is made from a shape-memory material.

Example 78. The delivery assembly of any example herein, particularly of example 77, wherein the shape-memory material is Nitinol.

Example 79. The delivery assembly of any example herein, particularly of any one of examples 59 to 78, wherein the at least one positioning arm comprises a plurality of positioning arms.

Example 80. The delivery assembly of any example herein, particularly of any one of examples 59 to 79, wherein the at least one positioning arm comprises a radiopaque material.

Example 81. The delivery assembly of any example herein, particularly of any one of examples 59 to 80, wherein the delivery apparatus further comprises at least one tensioning member attached to the free end of the at least one positioning arm, and extending proximally therefrom.

Example 82. The delivery assembly of any example herein, particularly of example 81, wherein the positioning arm is configured to transition to the compacted state when the tensioning member attached thereto is tensioned.

Example 83. The delivery assembly of any example herein, particularly of example 81 or 82, wherein the tensioning member extends from the free end of the positioning arm it is attached to, through a corresponding side opening formed at the first capsule, into the first capsule.

Example 84. The delivery assembly of any example herein, particularly of example 81 or 82, wherein the tensioning member extends from the free end of the positioning arm it is attached to, through a corresponding side opening formed at the delivery shaft, into the delivery shaft.

Example 85. The delivery assembly of any example herein, particularly of any one of examples 81 to 84, wherein the tensioning member comprises at least one of: a wire, a string, a suture, and/or a cable.

Example 86. The delivery assembly of any example herein, particularly of any one of examples 59 to 85, wherein the delivery apparatus further comprises a nosecone shaft extending through the delivery shaft, and a nosecone attached to the nosecone shaft.

Example 87. The delivery assembly of any example herein, particularly of example 86, wherein the second capsule is coupled to the nosecone shaft.

Example 88. The delivery assembly of any example herein, particularly of example 86, wherein the second capsule is coupled to the nosecone.

Example 89. The delivery assembly of any example herein, particularly of example 88, wherein the second capsule is integrally formed with the nosecone.

Example 90. A method of implanting a prosthetic valve, comprising:

advancing a delivery assembly that comprises a delivery apparatus carrying a prosthetic valve having a frame retained in a radially compressed state inside a first capsule and a second capsule of the delivery apparatus, to a native heart valve, while one or more positioning arms attached at bases thereof to the first capsule are retained in a compacted state between the first capsule and a second capsule proximal portion disposed over the positioning arms;

uncovering the positioning arms from the second capsule proximal portion, such that free ends of the positioning arms spring outwardly towards Valsalva sinuses of the native heart valve, while at least a portion of an outflow section of the prosthetic valve remains retained inside the first capsule;

adjusting the axial position of the first capsule such that the free ends of the positioning arms engage with sinus ceilings of the Valsalva sinuses; and

exposing the prosthetic valve such that it is allowed to expand against the native aortic valve.

Example 91. The method of any example herein, particularly of example 90, wherein the frame is movable between a radially compressed state and a radially expanded state.

Example 92. The method of any example herein, particularly of example 91, wherein the frame is self-expandable.

Example 93. The method of any example herein, particularly of any one of examples 90 to 92, wherein the base is affixed to a distal end portion of the first capsule.

Example 94. The method of any example herein, particularly of any one of examples 90 to 93, wherein the second capsule further comprises a second capsule distal portion.

Example 95. The method of any example herein, particularly of example 94, wherein an inner diameter defined by the second capsule proximal portion is greater than an inner diameter defined by the second capsule distal portion.

Example 96. The method of any example herein, particularly of example 94 or 95, wherein the advancing the delivery assembly comprises retaining at least part of an inflow section of the frame in the compressed state inside the second capsule distal portion.

Example 97. The method of any example herein, particularly of example 96, further comprising, after the uncovering the positioning arms, exposing at least a portion of the inflow section from the second capsule.

Example 98. The method of any example herein, particularly of example 97, wherein the exposing the prosthetic valve comprises exposing the outflow section from the first capsule.

Example 99. The method of any example herein, particularly of any one of examples 90 to 98, wherein the positioning arms extend proximally from the bases to the free ends.

Example 100. The method of any example herein, particularly of any one of examples 90 to 99, wherein the uncovering the positioning arms comprises axially moving the second capsule and the first capsule away from each other.

Example 101. The method of any example herein, particularly of example 100, wherein the axially moving comprises positioning a proximal edge of the second capsule distal to a distal edge of the first capsule.

Example 102. The method of any example herein, particularly of any one of examples 90 to 101, wherein the adjusting the axial position of the first capsule comprises simultaneously moving the first capsule and the prosthetic valve relative to the native heart valve.

Example 103. The method of any example herein, particularly of any one of examples 90 to 102, wherein the adjusting the axial position of the first capsule comprises

Example 104. The method of any example herein, particularly of any one of examples 90 to 103, wherein the adjusting the axial position of the first capsule comprises positioning the bases of the one or more positioning arms proximal to native leaflets of the native heart valve.

Example 105. The method of any example herein, particularly of any one of examples 90 to 104, wherein the adjusting the axial position comprises pressing the free ends against the sinus ceilings.

Example 106. The method of any example herein, particularly of any one of examples 90 to 105, wherein the free ends are atraumatic.

Example 107. The method of any example herein, particularly of any one of examples 96 to 98, wherein the exposing the prosthetic valve comprises anchoring the inflow section against a native annulus of the native heart valve.

Example 108. The method of any example herein, particularly of any one of examples 90 to 107, wherein the exposing the prosthetic valve comprises anchoring the outflow section against an ascending aorta proximal to the Valsalva sinuses.

Example 109. The method of any example herein, particularly of any one of examples 90 to 108, wherein the delivery apparatus further comprises a delivery shaft having the first capsule at a distal end thereof, a nosecone shaft extending through and axially movable relative to the delivery shaft, and a nosecone attached to the nosecone shaft.

Example 110. The method of any example herein, particularly of example 109, wherein the second capsule is coupled to the nosecone shaft and is axially movable therewith.

Example 111. The method of any example herein, particularly of example 109, wherein the second capsule is coupled to the nosecone and is axially movable therewith.

Example 112. The method of any example herein, particularly of any one of examples 90 to 111, further comprising, after the adjusting the axial position of the second capsule and prior to the exposing the prosthetic valve, transitioning the positioning arms to the compacted state.

Example 113. The method of any example herein, particularly of example 112, wherein the transitioning the positioning arms to the compacted state comprises applying tension to tensioning members coupled to the free ends of the positioning arms and extending proximally therefrom.

Example 114. The method of any example herein, particularly of example 112 or 113, wherein the exposing the prosthetic valve comprises, subsequent to the transitioning the positioning arms to the compacted state, proximally pulling the first capsule relative to the prosthetic valve.

Example 115. The method of any example herein, particularly of any one of examples 90 to 114, wherein the exposing the prosthetic valve comprises anchoring the outflow section against an ascending aorta proximal to the Valsalva sinuses.

Example 116. The method of any example herein, particularly of any one of examples 90 to 115, wherein the prosthetic valve further comprises a valvular structure mounted within the frame, the valvular structure comprising a plurality of leaflets configured to regulate flow through the prosthetic valve.

Example 117. The method of any example herein, particularly of example 116, wherein, after the exposing the prosthetic valve, an axial distance of at least 2 mm. is defined between an STJ level of the native heart valve and the valvular structure of the prosthetic valve.

Example 118. The method of any example herein, particularly of example 116, wherein, after the exposing the prosthetic valve, an axial distance of at least 2 mm. is defined between an STJ level of the native heart valve and free edges of the leaflets of the valvular structure.

Example 119. The method of any example herein, particularly of example 116, wherein, after the exposing the prosthetic valve, an axial distance of at least 2 mm. is defined between an STJ level of the native heart valve and commissures of the valvular structure.

Example 120. The method of any example herein, particularly of any one of examples 90 to 116, wherein, after the exposing the prosthetic valve, an axial distance of at least 2 mm. is defined between an STJ level of the native heart valve and an inner skirt of the prosthetic valve.

Example 121. The method of any example herein, particularly of any one of examples 90 to 120, wherein the adjusting the axial position comprises using tactile feedback from the one or more positioning members to confirm engagement with the sinus ceilings.

Example 122. The method of any example herein, particularly of any one of examples 90 to 121, wherein the one or more positioning members is radiopaque, and wherein the adjusting the axial position comprises identifying, under image guidance, deformation of the one or more positioning members due to engagement with the sinus ceilings.

Example 123. The method of any example herein, particularly of any one of examples 90 to 122, further comprising, subsequent to the exposing, releasing the delivery apparatus from the prosthetic valve.

Example 124. The method of any example herein, particularly of example 123, wherein the releasing comprises decoupling the delivery apparatus from retaining elements of the prosthetic valve.

Example 125. The method of any example herein, particularly of example 123 or 124, further comprising, subsequent to the releasing, withdrawing the delivery apparatus from the native heart valve.

Example 126. The method of any example herein, particularly of any one of examples 90 to 125, wherein the native heart valve is a native aortic valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a frame movable between a radially compressed state and a radially expanded state;
a valvular structure mounted within the frame and comprising a plurality of leaflets configured to regulate flow through the prosthetic valve; and
at least one positioning arm extending proximally from a base affixed to the frame, to a free end, wherein the at least one positioning arm is configured to transition between a compacted state and a free state;
wherein the at least one positioning arm is biased to the free state in which the free end is radially spaced from the frame and is distal to a proximal end of the frame; and
wherein the free end is proximal to the valvular structure in the free state.

2. The prosthetic valve of claim 1, wherein the base is proximal to a distal end of the frame.

3. The prosthetic valve of claim 1, wherein each leaflet comprises a free edge, and wherein the free end is proximal to free edges of the leaflets.

4. The prosthetic valve of claim 3, wherein the axial distance between the free edge of the at least one positioning member and the free edges of the leaflets is greater than 2 mm.

5. The prosthetic valve of claim 1, wherein the valvular structure comprises commissures coupled to commissure attachment features of the frame, and wherein the free end is proximal to commissures in the free state.

6. The prosthetic valve of claim 5, wherein the axial distance between the free end of the at least one positioning member and the commissures is greater than 2 mm.

7. The prosthetic valve of claim 1, further comprising an inner skirt disposed around the frame, wherein the free end is proximal to the inner skirt in the free state.

8. The prosthetic valve of claim 7, wherein the axial distance between the free end of the at least one positioning member and the inner skirt is greater than 2 mm.

9. The prosthetic valve of claim 1, wherein the free end is atraumatic.

10. The prosthetic valve of claim 1, wherein the free end is configured to engage with a sinus ceiling of a Valsalva sinus.

11. The prosthetic valve of claim 10, wherein, when the free end is engaged with the sinus ceiling, an axial gap is defined between an STJ level and the valvular structure.

12. The prosthetic valve of claim 11, wherein the axial gap is greater than 2 mm.

13. A method of implanting a prosthetic valve, comprising:
advancing a delivery assembly that comprises a delivery apparatus carrying a prosthetic valve retained in a radially compressed state inside a capsule of the delivery apparatus, to a native heart valve;
exposing at least a portion of an inflow section of the prosthetic valve and one or more positioning arms affixed to a frame of the prosthetic valve, such that free ends of the positioning arms spring outwardly towards Valsalva sinuses of the native heart valve, while at least a portion of an outflow section of the prosthetic valve remains retained inside the capsule;
adjusting the axial position of the prosthetic valve such that the free ends of the positioning arms engage with sinus ceilings of the Valsalva sinuses; and
exposing the remainder of the prosthetic valve from the capsule.

14. The method of claim 13, wherein each of the one or more positioning arms extends proximally from a base at which it is affixed to the frame, to the free end.

15. The method of claim 14, wherein the adjusting the axial position comprises positioning the bases of the one or more positioning arms proximal to a native annulus of the native heart valve.

16. The method of claim 13, wherein the adjusting the axial position comprises pressing the free ends against the sinus ceilings.

17. The method of claim 13, wherein the prosthetic valve further comprises a valvular structure mounted within the frame, the valvular structure comprising a plurality of leaflets configured to regulate flow through the prosthetic valve.

18. The method of claim 17, wherein, when the free ends are engaged with the sinus ceilings, an axial distance of at least 2 mm. is defined between the free ends and the valvular structure.

19. The method of claim 13, wherein the adjusting the axial position comprises using tactile feedback from the one or more positioning members to confirm engagement with the sinus ceilings.

20. The method of claim 13, wherein the one or more positioning members is radiopaque, and wherein the adjusting the axial position comprises identifying, under image guidance, deformation of the one or more positioning members due to engagement with the sinus ceilings.
